(19) United States
(12) Patent Application Publication
(10) Pub. No.: US 2008/0194554 A1
(43) Pub. Date: Aug. 14, 2008

(54) Hiv Protease Inhibitors

(76) Inventors: Ed W. McLean, Triangle Park, NC (US); John Franklin Miller, Triangle Park, NC (US)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 11/908,305

(22) PCT Filed: Mar. 7, 2006
(86) PCT No.: PCT/US06/08102

§ 371 Date: Sep. 11, 2007

Related U.S. Application Data
(60) Provisional application No. 60/660,706, filed on Mar. 11, 2005.

(51) Int. Cl. A61K 31/535; A61K 31/425; A61K 31/415; A61K 31/44

(52) U.S. Cl. 514/233.8; 514/365; 514/338; 514/397; 514/407

(57) ABSTRACT

The present invention features compounds that are HIV protease inhibitors and therefore are useful in the inhibition of HIV replication, the prevention and/or treatment of infection by HIV, and in the treatment of AIDS and/or ARC.

The human immunodeficiency virus (“HIV”) is the causative agent for acquired immunodeficiency syndrome (“AIDS”), a disease characterized by the destruction of the immune system, particularly of CD4⁺ T-cells, with attendant susceptibility to opportunistic infections, and its precursor AIDS-related complex (“ARC”), a syndrome characterized by symptoms such as persistent generalized lymphadenopathy, fever and weight loss.

As in the case of several other retroviruses, HIV encodes the production of a protease which carries out post-translational cleavage of precursor polypeptides in a process necessary for the formation of infectious virions (S. Crawford et al., “A Deletion Mutation in the 5′ Part of the pol Gene of Moloney Murine Leukemia Virus Blocks Proteolytic Processing of the gag and pol Polyproteins”, J. Virol., 53, p. 899 (1985)). These gene products include pol, which encodes the virion RNA-dependent DNA polymerase (reverse transcriptase), an endonuclease, HIV protease, and gag, which encodes the core-proteins of the virion (H. Toh et al., “Close Structural Resemblance Between Putative Polymerase of a Drosophila Transposable Genetic Element 17.6 and pol gene product of Moloney Murine Leukemia Virus”, EMBO J., 4, p. 1267 (1985); L. H. Pearl et al., “A Structural Model for the Retroviral Proteases”, Nature, pp. 329-351 (1987); M. D. Power et al., “Nucleotide Sequence of SRV-1, a Type D Simian Acquired Immune Deficiency Syndrome Retrovirus”, Science, 231, p. 1567 (1986)).

A number of synthetic anti-viral agents have been designed to target various stages in the replication cycle of HIV. These agents include compounds which block viral binding to CD4⁺ T-lymphocytes (for example, soluble CD4), and compounds which interfere with viral replication by inhibiting viral reverse transcriptase (for example, didanosine and zidovudine (AZT)) and inhibit integration of viral DNA into cellular DNA (M. S. Hirsh and R. T. D'Aqulia, “Therapy for Human Immunodeficiency Virus Infection”, N. Eng. J. Med., 328, p. 1686 (1993)). However, such agents, which are directed primarily to early stages of viral replication, do not prevent the production of infectious virions in chronically infected cells. Furthermore, administration of some of these agents in effective amounts has led to cell-toxicity and unwanted side effects, such as anemia and bone marrow suppression.

More recently, the focus of anti-viral drug design has been to create compounds which inhibit the formation of infectious virions by interfering with the processing of viral polyprotein precursors. Processing of these precursor proteins requires the action of virus-encoded proteases which are essential for replication (Kohl, N. E. et al. “Active HIV Protease is Required for Viral Infectivity” Proc. Natl. Acad. Sci. USA, 85, p. 4686 (1988)). The anti-viral potential of HIV protease inhibition has been demonstrated using peptidyl inhibitors.

More recently several small molecule protease inhibitors have become available for the treatment of HIV infections. Among these is the sulfonamide-containing molecule, Agenerase®. Agenerase® is described in U.S. Pat. No. 5,585,397. Other sulfonamide inhibitors of aspartyl protease are described in U.S. Pat. Nos. 5,691,372, 5,510,388, 5,521,219, 5,639,769, 5,714,605, 5,744,481, 5,786,483, 5,830,897 and 5,843,946.

Because HIV-infected patients often develop resistance to particular protease inhibitors, the need still exists for additional compounds that can effectively inhibit the action of aspartyl proteases, particularly HIV protease, for use as agents for preventing and treating chronic and acute viral infections. WO 00/76961 discloses inhibitors of aspartyl protease, including N-(3R,3aS,6aR)-hexahydrofuro[2,3-b]furan-3-yl-oxycarbonyl-, (4S,5R)-4-[4-(2-methylthiazolo-4-methyloxy)-benzyl]-5-i-butyl-[(3,4-methylenedioxyphenyl)sulfonyl]-aminomethyl-2,2-dimethyl-oxazolidine (also known as GW0385) which is currently under development for the treatment of HIV infection. The compounds of the present invention may serve as prodrugs of GW0385.

The present invention features compounds of formula (I)

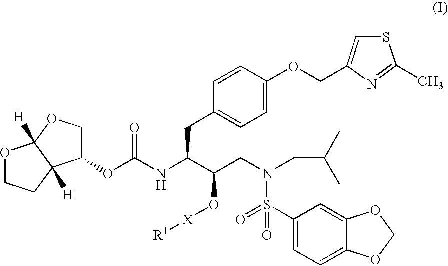

wherein:

X is a C₁₋₅ alkylene chain, wherein said C₁₋₅ alkylene chain is optionally substituted by one or more groups selected from ═O, ═N, —NH₂, and —C₁₋₈alkyl and wherein said C₁₋₅ alkylene chain optionally contains 1-4 heteroatoms selected from oxygen, sulfur and nitrogen wherein such heteroatom is optionally substituted with one or more groups selected from hydrogen and C₁₋₈alkyl;

R¹ is amino, C₁₋₈alkyl, C₁₋₈alkoxy, —NR₂, —N(R²)₂, or heterocycle optionally substituted with C₁₋₈alkyl;

R² is C₁₋₈alkyl or C₁₋₈alkoxy;

or a pharmaceutically acceptable derivative thereof.

The present invention also features compounds of formula (I) wherein —X—R¹ is selected from the group consisting of:

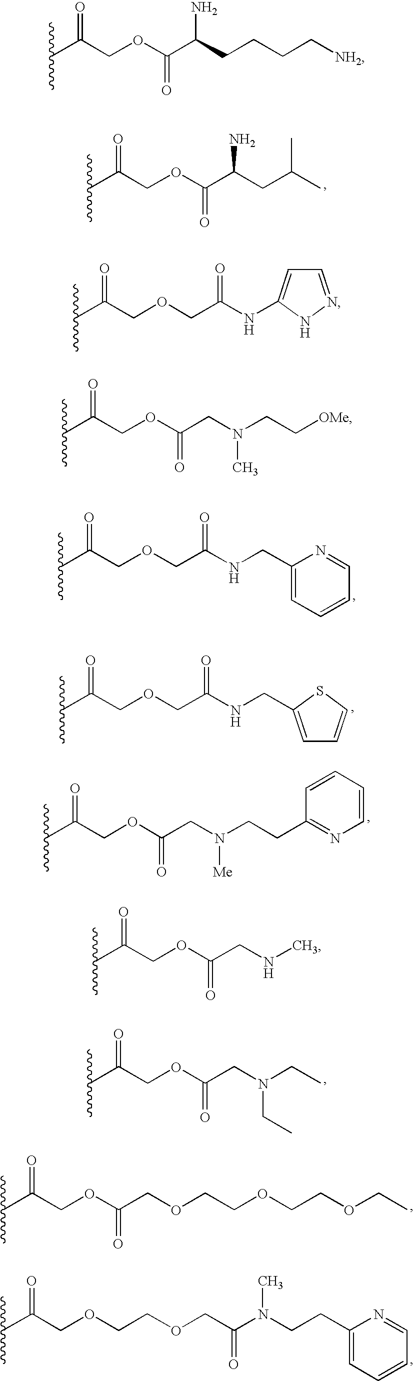

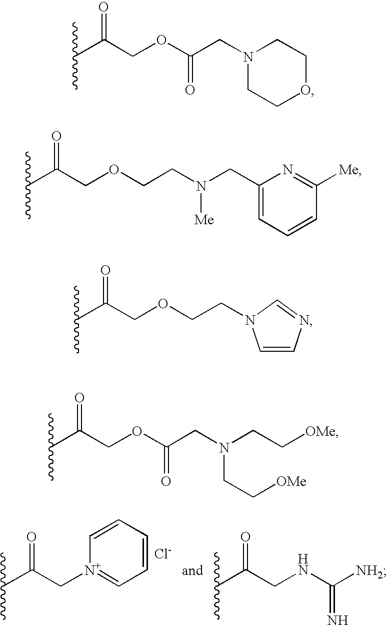

and pharmaceutically acceptable derivatives thereof.

The term “alkyl”, alone or in combination with any other term, refers to a straight-chain or branched-chain saturated aliphatic hydrocarbon radical containing the specified number of carbon atoms. Examples of alkyl radicals include, but are not limited to, methyl, ethyl, n-propyl, isopropyl, n-butyl, isobutyl, sec-butyl, tert-butyl, pentyl, isoamyl, n-hexyl and the like.

The term “alkylene chain” refers to a straight or branched hydrocarbon chain that may be fully saturated or have one or more units of unsaturation. The unsaturation may occur in any stable point along the chain. The double bond(s) in the unsaturated alkylene chain may be in either the cis or trans configuration.

The term “alkoxy” refers to an alkyl ether radical, wherein the term “alkyl” is defined above. Examples of suitable alkyl ether radicals include, but are not limited to, methoxy, ethoxy, n-propoxy, isopropoxy, n-butoxy, isobutoxy, sec-butoxy, tert-butoxy and the like.

The term “heterocycle,” “heterocyclic,” and “heterocyclyl” as used herein, refer to a 3- to 7-membered monocyclic heterocyclic ring or 8- to 11-membered bicyclic heterocyclic ring system any ring of which is either saturated, partially saturated or unsaturated, and which may be optionally benzofused if monocyclic. Each heterocycle consists of one or more carbon atoms and from one to four heteroatoms selected from the group consisting of N, O and S, and wherein the nitrogen and sulfur heteroatoms may optionally be oxidized, and the nitrogen atom may optionally be quaternized, and including any bicyclic group in which any of the above-defined heterocyclic rings is fused to a benzene ring. The heterocyclic ring may be attached at any carbon or heteroatom, provided that the attachment results in the creation of a stable structure. Preferred heterocycles include 5-7 membered monocyclic heterocycles and 8-10 membered bicyclic heterocycles. When the heterocyclic ring has substituents, it is understood that the substituents may be attached to any atom in the ring, whether a heteroatom or a carbon atom, provided that a stable chemical structure results. “Heteroaromatics” or “heteroaryl” are included within the heterocycles as defined above and generally refers to a heterocycle in which the ring system is an aromatic monocyclic or polycyclic ring radical containing five to twenty carbon atoms, preferably five to ten carbon atoms, in which one or more ring carbons, preferably one to four, are each replaced by a heteroatom such as N, O, S and P. Preferred heteroaryl groups include 5-6 membered monocyclic heteroaryls and 8-10 membered bicyclic heteroaryls. Also included within the scope of the term “heterocycle, “heterocyclic” or “heterocyclyl” is a group in which a non-aromatic heteroatom-containing ring is fused to one or more aromatic rings, such as in an indolinyl, chromanyl, phenanthridinyl or tetrahydro-quinolinyl, where the radical or point of attachment is on the non-aromatic heteroatom-containing ring. Unless otherwise indicated, the term “heterocycle, “heterocyclic” or “heterocyclyl” also included each possible positional isomer of a heterocyclic radical, such as in 1-indolinyl, 2-indolinyl, 3-indolinyl. Examples of heterocycles include imidazolyl, imidazolinoyl, imidazolidinyl, quinolyl, isoquinolyl, indolyl, indazolyl, indazolinolyl, perhydropyridazyl, pyridazyl, pyridyl, pyrrolyl, pyrrolinyl, pyrrolidinyl, pyrazolyl, pyrazinyl, quinoxolyl, piperidinyl, pyranyl, pyrazolinyl, piperazinyl, pyrimidinyl, pyridazinyl, morpholinyl, thiamorpholinyl, furyl, thienyl, triazolyl, thiazolyl, carbolinyl, tetrazolyl, thiazolidinyl, benzofuranoyl, thiamorpholinyl sulfone, oxazolyl, oxadiazolyl, benzoxazolyl, oxopiperidinyl, oxopyrrolidinyl, oxoazepinyl, azepinyl, isoxozolyl, isothiazolyl, furazanyl, tetrahydropyranyl, tetrahydrofuranyl, thiazolyl, thiadiazoyl, dioxolyl, dioxinyl, oxathiolyl, benzodioxolyl, dithiolyl, thiophenyl, tetrahydrothiophenyl, sulfolanyl, dioxanyl, dioxolanyl, tetahydrofurodihydrofuranyl, tetrahydropyranodihydrofuranyl, dihydropyranyl, tetradyrofurofuranyl and tetrahydropyranofuranyl.

The term “heteroatom” means nitrogen, oxygen, or sulfur and includes any oxidized form of nitrogen, such as N(O) {N⁺—O⁻} and sulfur such as S(O) and S(O)₂, and the quaternized form of any basic nitrogen.

A combination of substituents or variables is permissible only if such a combination results in a stable or chemically feasible compound. A stable compound or chemically feasible compound is one in which the chemical structure is not substantially altered when kept at a temperature of 40° C. or less, in the absence of moisture or other chemically reactive conditions, for at least a week.

Unless otherwise stated, structures depicted herein are also meant to include all stereochemical forms of the structure, i.e., the R and S configurations for each asymmetric center. Therefore, racemates and racemic mixtures, single enantiomers, diastereomeric mixtures and individual diastereoisomers of the present compounds are expressly included within the scope of the invention. Although the specific compounds exemplified herein may be depicted in a particular stereochemical configuration, compounds having either the opposite stereochemistry at any given chiral center or mixtures thereof are also envisioned.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C— or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other.

The term “pharmaceutically effective amount” refers to an amount effective in treating a virus infection, for example an HIV infection, in a patient either as monotherapy or in combination with other agents. The term “treating” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. The term “prophylactically effective amount” refers to an amount effective in preventing a virus infection, for example an HIV infection, or preventing the occurrence of symptoms of such an infection, in a patient. As used herein, the term “patient” refers to a mammal, including a human.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the antiviral agent.

The term “treatment” as used herein refers to the alleviation of symptoms of a particular disorder in a patient, or the improvement of an ascertainable measurement associated with a particular disorder, and may include the suppression of symptom recurrence in an asymptomatic patient such as a patient in whom a viral infection has become latent. Treatment includes prophylaxis which refers to preventing a disease or condition or preventing the occurrence of symptoms of such a disease or condition, in a patient. As used herein, the term “patient” refers to a mammal, including a human.

As used herein, the term “subject” refers to a patient, animal or a biological sample. The term “biological sample”, as used herein, includes, without limitation, cell cultures or extracts thereof; preparations of an enzyme suitable for in vitro assay; biopsied material obtained from a mammal or extracts thereof; and blood, saliva, urine, feces, semen, tears, or other body fluids or extracts thereof.

Throughout this specification, the word “comprise” or variations such as “comprises” or “comprising” will be understood to imply the inclusion of a stated integer or groups of integers but not the exclusion of any other integer or group of integers.

As used herein, the compounds according to the invention are defined to include pharmaceutically acceptable derivatives thereof. A “pharmaceutically acceptable derivative” means any pharmaceutically acceptable salt, ester, salt of an ester, ether, or other derivative of a compound of this invention which, upon administration to a recipient, is capable of providing directly or indirectly a compound of this invention or an inhibitorily active metabolite or residue thereof. Particularly favored derivatives and prodrugs are those that increase the bioavailability of the compounds of this invention when such compounds are administered to a mammal, for example, by allowing an orally administered compound to be more readily absorbed into the blood, or which enhance delivery of the parent compound to a biological compartment, for example, the brain or lymphatic system, relative to the parent species.

The present invention further features compounds of formula (II)

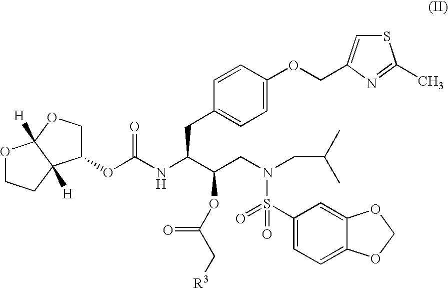

wherein:

R³ is hydroxy, halogen, aminoC₁₋₈alkyl, heterocycle, heterocycle C₁₋₈alkyl, N(R⁴)R⁵, NHR⁵, NHR⁵, OR⁵, OR⁶R⁷, OC(O)R⁴, OC(O)R⁵, OC(O)R⁶, OC(O)R⁴NR⁴, OC(O)R⁴NHR⁴, OC(O)NR⁴OR⁴, OC(O)R⁴N(R⁴)R⁵, OC(O)R⁴NHC(O)OR⁴, OC(O)R⁴N(R⁴)C(O)OR⁴, OC(O)R⁴N(R⁵)R⁴OR⁴, OR⁴C(O)OH, OR⁴C(O)NHR⁷, OR⁶C(O)N(R⁴)R⁷, OR⁶OC(O)OH;

R⁴ is C₁₋₈alkyl;

R⁵ is C₁₋₈alkyl, optionally substituted with one or more substituents selecting from the group consisting of amino, ═NH, N₃, halogen, oxo, C₁₋₈alkoxy, heterocycle, heterocyclealkyl, or NHC(O)R⁴; each of which may be optionally substituted with C₁₋₈alkyl;

R⁶ is C₁₋₅ alkylene chain, wherein said C₁₋₅ alkylene chain is optionally substituted by one or more groups selected from ═O, ═N, —NH₂, and —C₁₋₈alkyl and wherein said C₁₋₅ alkylene chain optionally contains 1-4 heteroatoms selected from oxygen, sulfur and nitrogen wherein such heteroatom is optionally substituted with one or more groups selected from hydrogen and C₁₋₈alkyl;

R⁷ is heterocycle optionally substituted with C₁₋₈alkyl or heterocycleC₁₋₈alkyl;

or a pharmaceutically acceptable derivative thereof.

The present invention features a compound selected from the group consisting of:

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(2-methoxyethyl)-N-methylglycinate;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methyl-N-(2-pyridin-2-ylethyl)glycinate;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-diethylglycinate;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl morpholin-4-ylacetate;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-bis(2-methoxyethyl)glycinate;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-lysinate tris-trifluoroacetic acid salt;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-leucinate bis-trifluoroacetic acid salt;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methylglycinate bis-trifluoroacetic acid salt;

(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-(1H-imidazol-1-yl)ethoxy]acetate bis-trifluoroacetic acid salt;

2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl[2-(2-ethoxyethoxy)ethoxy]acetate;

(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{methyl[(6-methylpyridin-2-yl)methyl]amino}ethoxy)acetate tris-trifluoroacetic acid salt;

(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-oxo-2-(1H-pyrazol-5-ylamino)ethoxy]acetate;

(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl{2-oxo-2-[(pyridin-2-ylmethyl)amino]ethoxy}acetate;

(1R,2S)-2-({[(3R,3aS,6aR)-Hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl{2-oxo-2-[(thien-2-ylmethyl)amino]ethoxy}acetate;

(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{2-[methyl(2-pyridin-2-ylethyl)amino]-2-oxoethoxy}ethoxy)acetate;

(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl N-[amino(imino)methyl]glycinate tris-trifluoroacetic acid salt;

1-{2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl}pyridinium chloride;

and pharmaceutically acceptable derivatives thereof.

The compounds of the present invention can be readily prepared by techniques known in the art. Schemes I and II illustrate general synthetic routes to the compounds of this invention.

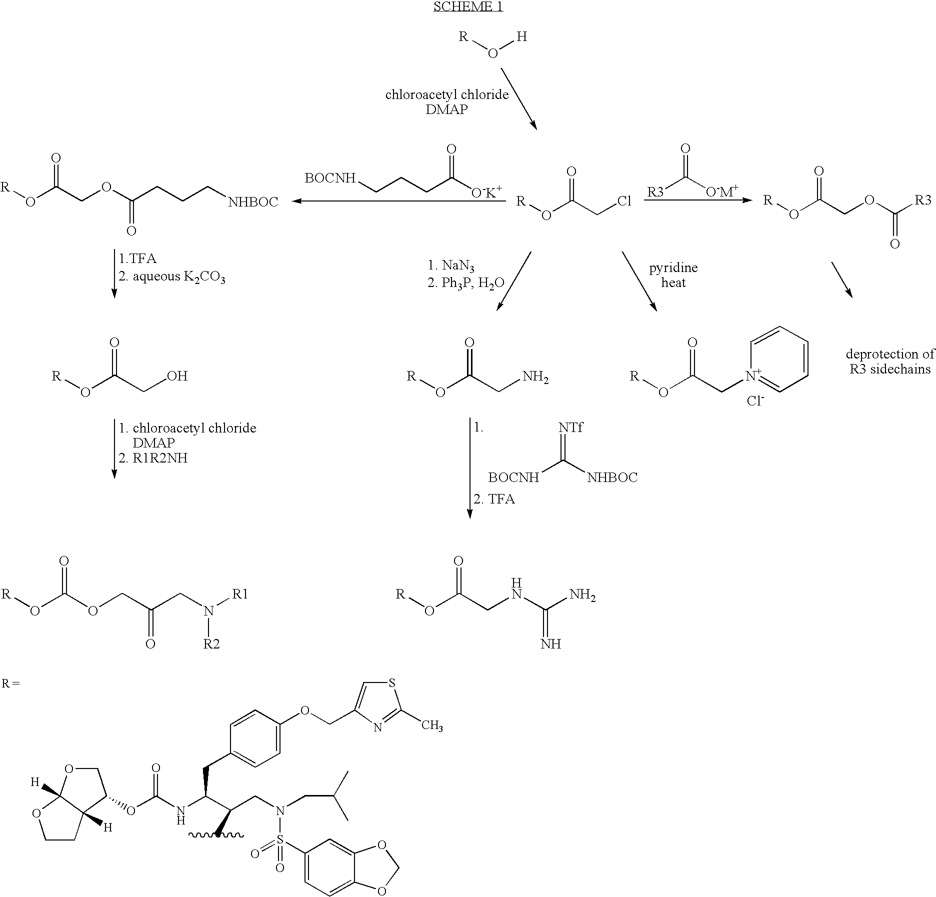

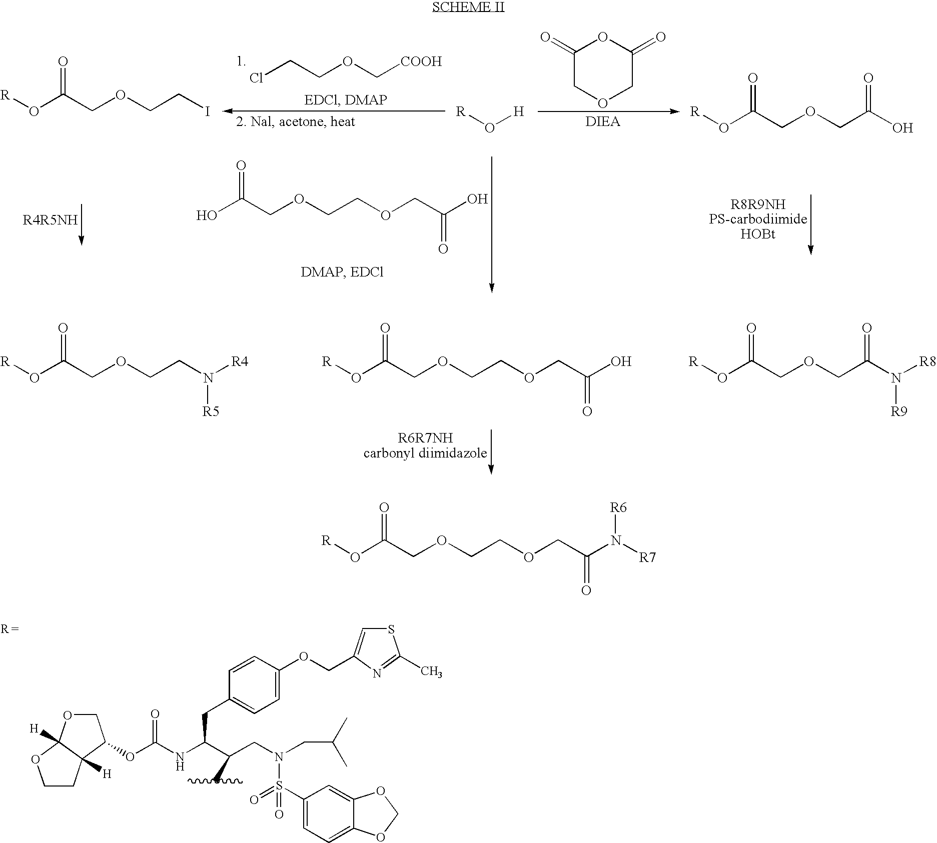

Thus, the synthetic approaches illustrated in schemes I and II can be readily extended to produce other compounds of the present invention. The above synthetic schemes are not intended to comprise a comprehensive list of all means by which compounds described and claimed in this application may be synthesized. Further methods will be evident to those of ordinary skill in the art.

Compounds of the present invention demonstrate advantageous pharmaceutical properties in that they have features which result in increased solubility, for example, side chains that are either polar or that enhance solubility through ease of salt formation.

Pharmaceutically acceptable salts of the compounds according to the invention include those derived from pharmaceutically acceptable inorganic and organic acids and bases. Examples of suitable acids include hydrochloric, hydrobromic, sulfuric, nitric, perchloric, fumaric, maleic, phosphoric, glycollic, lactic, salicyclic, succinic, toluene-p-sulfonic, tartaric, acetic, citric, methanesulfonic, ethanesulfonic, formic, benzoic, malonic, naphthalene-2-sulfonic and benzenesulfonic acids. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g. sodium), alkaline earth metal (e.g., magnesium), ammonium, NW₄⁺ (wherein W is C₁₋₄ alkyl) and other amine salts. Physiologically acceptable salts of a hydrogen atom or an amino group include salts or organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids. Physiologically acceptable salts of a compound with a hydroxy group include the anion of said compound in combination with a suitable cation such as Na⁺, NH₄⁺, and NW₄⁺ (wherein W is a C₁₋₄alkyl group). Preferred salts include sodium, calcium, potassium, magnesium, choline, meglumine, hydrochloride, and quaternary ammonium. The invention features pharmaceutically acceptable salts of the compounds of formula (I) and (II).

Other compounds of this invention may be prepared by one skilled in the art following the teachings of the specification coupled with knowledge in the art using reagents that are readily synthesized or commercially available.

Any reference to any of the above compounds also includes a reference to a pharmaceutically acceptable salt thereof.

Salts of the compounds of the present invention may be made by methods known to a person skilled in the art. For example, treatment of a compound of the present invention with an appropriate base or acid in an appropriate solvent will yield the corresponding salt.

Esters of the compounds of the present invention are independently selected from the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted by, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

In such esters, unless otherwise specified, any alkyl moiety present advantageously contains from 1 to 18 carbon atoms, particularly from 1 to 6 carbon atoms, more particularly from 1 to 4 carbon atoms. Any cycloalkyl moiety present in such esters advantageously contains from 3 to 6 carbon atoms. Any aryl moiety present in such esters advantageously comprises a phenyl group.

Ethers of the compounds of the present invention include, but are not limited to methyl, ethyl, butyl and the like.

A combination of substituents or variables is permissible only if such a combination results in a stable or chemically feasible compound. A stable compound or chemically feasible compound is one in which the chemical structure is not substantially altered when kept at a temperature of 40° C. or less, in the absence of moisture or other chemically reactive conditions, for at least a week.

Compounds of the present invention are useful as prodrugs of HIV protease inhibitors. One aspect of the instant invention relates to methods of treating or preventing viral infection, for example an HIV infection, in a biological sample comprising contacting the biological sample with compounds of formula (I) or (II) or pharmaceutically acceptable derivatives thereof. Another aspect of the instant invention relates to methods of treating or preventing viral infection, for example, an HIV infection, in a patient comprising administering to the patient a therapeutically effective amount of compounds of formula (I) or (II) or pharmaceutically acceptable derivatives thereof.

The compounds according to the invention are particularly suited to the treatment or prophylaxis of HIV infections and associated conditions. Reference herein to treatment extends to prophylaxis as well as the treatment of established infections, symptoms, and associated clinical conditions such as AIDS related complex (ARC), Kaposi's sarcoma, and AIDS dementia.

The compounds of the present invention may exhibit advantages over previously disclosed protease inhibitors, for example increased potency, metabolic stability, increased therapeutic index, or other pharmaceutical properties.

According to one embodiment of the invention, compounds of formula (I) or (II) or salts thereof may be formulated into compositions. In a preferred embodiment, the composition is a pharmaceutical composition, which comprises a compound of formula (I) or (II) and pharmaceutically acceptable carrier, adjuvant or vehicle. In one embodiment, the composition comprises an amount of a compound of the present invention effective to treat or prevent viral infection, for example an HIV infection, in a biological sample or in a patient. In another embodiment, compounds of this invention and pharmaceutical compositions thereof, which comprise an amount of a compound of the present innovation effective to inhibit viral replication or to treat or prevent a viral infection or disease or disorder, for example an HIV infection, and a pharmaceutically acceptable carrier, adjuvant or vehicle, may be formulated for administration to a patient, for example, for oral administration.

The present invention features compounds according to the invention for use in medical therapy, for example for the treatment or prophylaxis of a viral infection, for example an HIV infection and associated conditions. The compounds according to the invention are especially useful for the treatment of AIDS and related clinical conditions such as AIDS related complex (ARC), progressive generalized lymphadenopathy (PGL), Kaposi's sarcoma, thromobocytopenic purpura, AIDS-related neurological conditions such as AIDS dementia complex, multiple sclerosis or tropical paraperesis, anti-HIV antibody-positive and HIV-positive conditions, including such conditions in asymptomatic patients.

According to another aspect, the present invention provides a method for the treatment or prevention of the symptoms or effects of a viral infection in an infected patient, for example, a mammal including a human, which comprises administering to said patient a pharmaceutically effective amount of a compound according to the invention. According to one aspect of the invention, the viral infection is a retroviral infection, in particular an HIV infection.

The present invention further includes the use of a compound according to the invention in the manufacture of a medicament for administration to a subject for the treatment of a viral infection, in particular and HIV infection.

The compounds according to the invention may also be used in adjuvant therapy in the treatment of HIV infections or HIV-associated symptoms or effects, for example Kaposi's sarcoma.

The present invention further provides a method for the treatment of a clinical condition in a patient, for example, a mammal including a human which clinical condition includes those which have been discussed hereinbefore, which comprises treating said patient with a pharmaceutically effective amount of a compound according to the invention. The present invention also includes a method for the treatment or prophylaxis of any of the aforementioned diseases or conditions.

Reference herein to treatment extends to prophylaxis as well as the treatment of established conditions, disorders and infections, symptoms thereof, and associated. The above compounds according to the invention and their pharmaceutically acceptable derivatives may be employed in combination with other therapeutic agents for the treatment of the above infections or conditions. Combination therapies according to the present invention comprise the administration of a compound of the present invention or a pharmaceutically acceptable derivative thereof and another pharmaceutically active agent. The active ingredient(s) and pharmaceutically active agents may be administered simultaneously (i.e., concurrently) in either the same or different pharmaceutical compositions or sequentially in any order. The amounts of the active ingredient(s) and pharmaceutically active agent(s) and the relative timings of administration will be selected in order to achieve the desired combined therapeutic effect.

Examples of such therapeutic agents include, but are not limited to, agents that are effective for the treatment of viral infections or associated conditions. Among these agents are (1-alpha,2-beta,3-alpha)-9-[2,3-bis(hydroxymethyl)cyclobutyl]guanine [(−)BHCG, SQ-34514, lobucavir]; 9-[(2R,3R,4S)-3,4-bis(hydroxy methyl)2-oxetanosyl]adenine (oxetanocin-G); acyclic nucleosides, for example acyclovir, valaciclovir, famciclovir, ganciclovir, and penciclovir; acyclic nucleoside phosphonates, for example (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethyl propanoic acid (bis-POM PMEA, adefovir dipivoxil), [[(1R)-2-(6-amino-9H-purin-9-yl)-1-methylethoxy]methyl]phosphonic acid (tenofovir), and (R)-[[2-(6-Amino-9H-purin-9-yl)-1-methylethoxy]methyl]phosphonic acid bis-(isopropoxycarbonyloxymethyl)ester (bis-POC-PMPA); ribonucleotide reductase inhibitors, for example 2-acetylpyridine 5-[(2-chloroanilino)thiocarbonyl)thiocarbonohydrazone and hydroxyurea; nucleoside reverse transcriptase inhibitors, for example 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-azido-2′,3′-dideoxythymidine-5′-H-phosphophonate (phosphonovir), 2′-deoxy-5-iodo-uridine (idoxuridine), (−)-cis-1-(2-hydroxymethyl)-1,3-oxathiolane5-yl)-cytosine (lamivudine), cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclo-propylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin; protease inhibitors, for example indinavir, ritonavir, nelfinavir, amprenavir, saquinavir, fosamprenavir, (R)—N-tert-butyl-3-[(2S,3S)-2-hydroxy-3-N—[(R)-2-N-(isoquinolin-5-yloxyacetyl)amino-3-methylthio-propanoyl]amino-4-phenylbutanoyl]-5,5-dimethyl-1,3-thiazolidine-4-carboxamide (KNI-272), 4R-(4alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)—[N-(methoxycarbonyl)-1-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-hydroxy-1(S)-indanyl)-2(R)-phenyl-methyl-4(S)-hydroxy-5-(1-(1-(4-benzo[b]furanylmethyl)-2(S)-N′-(tert-butyl carboxamido)piperazinyl)pentanamide (MK-944A); interferons such as α-interferon; renal excretion inhibitors such as probenecid; nucleoside transport inhibitors such as dipyridamole, pentoxifylline, N-acetylcysteine (NAC), Procysteine, α-trichosanthin, phosphonoformic acid; as well as immunomodulators such as interleukin II or thymosin, granulocyte macrophage colony stimulating factors, erythropoetin, soluble CD₄ and genetically engineered derivatives thereof; non-nucleoside reverse transcriptase inhibitors (NNRTIs), for example nevirapine (BI-RG-587), alpha-((2-acetyl-5-methylphenyl)amino)-2,6-dichloro-benzeneacetamide (loviride), 1-[3-(isopropyl amino)-2-pyridyl]-4-[5-(methanesulfonamido)-1H-indol-2-ylcarbonyl]piperazine monomethanesulfonate (delavirdine), (10R,11S,12S)-12-Hydroxy-6,6,10,11-tetramethyl-4-propyl-11,12-dihydro-2H,6H,10H-benzo(1,2-b:3,4-b′:5,6-b″)tripyran-2-one ((+) calanolide A), (4S)-6-Chloro-4-[1E)-cyclopropyl ethenyl)-3,4-dihydro-4-(trifluoromethyl)-2(1H)-quinazolinone (DPC-083), (S)-6-chloro-4-(cyclopropyl ethynyl)-1,4-dihydro-4-(trifluoromethyl)-2H-3,1-benzoxazin-2-one (efavirenz, DMP 266), 1-(ethoxy methyl)-5-(1-methylethyl)-6-(phenylmethyl)-2,4(1H,3H)-pyrimidinedione (MKC-442), and 5-(3,5-dichloro phenyl)thio-4-isopropyl-1-(4-pyridyl)methyl-1H-imidazol-2-ylmethyl carbamate (capravirine); glycoprotein 120 antagonists, for example PRO-2000, PRO-542 and 1,4-bis[3-[(2,4-dichlorophenyl)carbonyl amino]-2-oxo-5,8-disodiumsulfanyl]naphthalyl-2,5-dimethoxyphenyl-1,4-dihydrazone (FP-21399); cytokine antagonists, for example reticulose (Product-R), 1,1′-azobis-formamide (ADA), 1,11-(1,4-phenylenebis(methylene))bis-1,4,8,11-tetraazacyclotetradecane octahydrochloride (AMD-3100); integrase inhibitors; and fusion inhibitors, for example T-20 and T-1249.

The present invention further includes the use of a compound according to the invention in the manufacture of a medicament for simultaneous or sequential administration with at least another therapeutic agent, such as those defined hereinbefore.

Compounds of the present invention may be administered with an agent known to inhibit or reduce the metabolism of compounds, for example ritonavir. Accordingly, the present invention features a method for the treatment or prophylaxis of a disease as hereinbefore described by administration of a compound of the present invention in combination with a metabolic inhibitor. Such combination may be administered simultaneously or sequentially.

In general a suitable dose for each of the above-mentioned conditions will be in the range of 0.01 to 250 mg per kilogram body weight of the recipient (e.g. a human) per day, preferably in the range of 0.1 to 100 mg per kilogram body weight per day and most preferably in the range 0.5 to 30 mg per kilogram body weight per day and particularly in the range 1.0 to 20 mg per kilogram body weight per day. Unless otherwise indicated, all weights of active ingredient are calculated as the parent compound of formula (I) or (II); for salts or esters thereof, the weights would be increased proportionally. The desired dose may be presented as one, two, three, four, five, six or more sub-doses administered at appropriate intervals throughout the day. In some cases the desired dose may be given on alternative days. These sub-doses may be administered in unit dosage forms, for example, containing 10 to 1000 mg or 50 to 500 mg, preferably 20 to 500 mg, and most preferably 50 to 400 mg of active ingredient per unit dosage form.

While it is possible for the active ingredient to be administered alone, it is preferable to present it as a pharmaceutical composition. The compositions of the present invention comprise at least one active ingredient, as defined above, together with one or more acceptable carriers thereof and optionally other therapeutic agents. Each carrier must be acceptable in the sense of being compatible with the other ingredients of the composition and not injurious to the patient.

Pharmaceutical compositions include those suitable for oral, rectal, nasal, topical (including transdermal, buccal and sublingual), vaginal or parenteral (including subcutaneous, intramuscular, intravenous, intradermal, and intravitreal) administration. The compositions may conveniently be presented in unit dosage form and may be prepared by any methods well known in the art of pharmacy. Such methods represent a further feature of the present invention and include the step of bringing into association the active ingredients with the carrier, which constitutes one or more accessory ingredients. In general, the compositions are prepared by uniformly and intimately bringing into association the active ingredients with liquid carriers or finely divided solid carriers or both, and then if necessary shaping the product.

The present invention further includes a pharmaceutical composition as hereinbefore defined wherein a compound of the present invention or a pharmaceutically acceptable derivative thereof and another therapeutic agent are presented separately from one another as a kit of parts.

Compositions suitable for transdermal administration may be presented as discrete patches adapted to remain in intimate contact with the epidermis of the recipient for a prolonged period of time. Such patches suitably contain the active compound 1) in an optionally buffered, aqueous solution or 2) dissolved and/or dispersed in an adhesive or 3) dispersed in a polymer. A suitable concentration of the active compound is about 1% to 25%, preferably about 3% to 15%. As one particular possibility, the active compound may be delivered from the patch by electrotransport or iontophoresis as generally described in Pharmaceutical Research 3(6), 318 (1986).

Pharmaceutical compositions of the present invention suitable for oral administration may be presented as discrete units such as capsules, caplets, cachets or tablets each containing a predetermined amount of the active ingredients; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or molding, optionally with one or more accessory ingredients. Compressed tablets may be prepared by compressing in a suitable machine the active ingredients in a free-flowing form such as a powder or granules, optionally mixed with a binder (e.g. povidone, gelatin, hydroxypropylmethyl cellulose), lubricant, inert diluent, preservative, disintegrant (e.g. sodium starch glycollate, cross-linked povidone, cross-linked sodium carboxymethyl cellulose) surface-active or dispersing agent. Molded tablets may be made by molding a mixture of the powdered compound moistened with an inert liquid diluent in a suitable machine. The tablets may optionally be coated or scored and may be formulated so as to provide slow or controlled release of the active ingredients therein using, for example, hydroxypropylmethyl cellulose in varying proportions to provide the desired release profile. Tablets may optionally be provided with an enteric coating, to provide release in parts of the gut other than the stomach.

Pharmaceutical compositions suitable for topical administration in the mouth include lozenges comprising the active ingredients in a flavored base, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions suitable for vaginal administration may be presented as pessaries, tampons, creams, gels, pastes, foams or spray. Pharmaceutical compositions may contain in addition to the active ingredient such carriers as are known in the art to be appropriate.

Pharmaceutical compositions for rectal administration may be presented as a suppository with a suitable carrier comprising, for example, cocoa butter or a salicylate or other materials commonly used in the art. The suppositories may be conveniently formed by admixture of the active combination with the softened or melted carrier(s) followed by chilling and shaping in molds.

Pharmaceutical compositions suitable for parenteral administration include aqueous and nonaqueous isotonic sterile injection solutions which may contain anti-oxidants, buffers, bacteriostats and solutes which render the pharmaceutical composition isotonic with the blood of the intended recipient; and aqueous and non-aqueous sterile suspensions which may include suspending agents and thickening agents; and liposomes or other microparticulate systems which are designed to target the compound to blood components or one or more organs. The pharmaceutical compositions may be presented in unit-dose or multi-dose sealed containers, for example, ampoules and vials, and may be stored in a freeze-dried (lyophilized) condition requiring only the addition of the sterile liquid carrier, for example water for injection, immediately prior to use. Extemporaneous injection solutions and suspensions may be prepared from sterile powders, granules and tablets of the kind previously described.

Unit dosage pharmaceutical compositions include those containing a daily dose or daily subdose of the active ingredients, as hereinbefore recited, or an appropriate fraction thereof.

It should be understood that in addition to the ingredients particularly mentioned above the pharmaceutical compositions of this invention may include other agents conventional in the art having regard to the type of pharmaceutical composition in question, for example, those suitable for oral administration may include such further agents as sweeteners, thickeners and flavoring agents.

The compounds of the present invention may be prepared according to the following reactions schemes and examples, or modifications thereof using readily available starting materials, reagents and conventional synthesis procedures.

### EXAMPLE 1

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(2-methoxyethyl)-N-methylglycinate

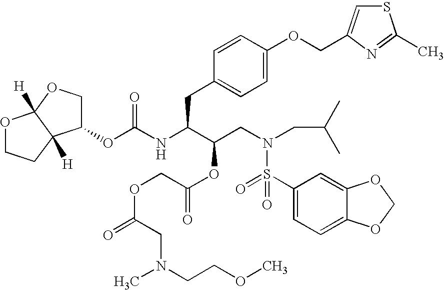

### Example 1, Step 1

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate

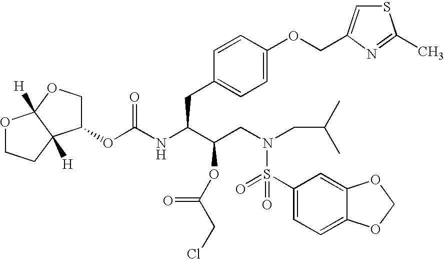

To a solution of (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (10.3 g, 14.6 mmol, see patent WO 2000076961 for preparation) and 4-(dimethylamino)pyridine (5.78 g, 47.3 mmol) in dichloromethane (260 mL) was added chloroacetyl chloride (3.50 mL, 43.7 mmol) dropwise over 15 minutes. The reaction mixture was stirred for 4 hours, then washed with 1N aqueous hydrochloric acid. The aqueous layer was washed with dichloromethane. The organic layers were combined and washed with water, saturated aqueous sodium bicarbonate and saturated aqueous sodium chloride. The solution was dried over magnesium sulfate and concentrated to a tan foam. Column chromatography on silica gel eluting with 5:4:1 ethyl acetate:hexane:triethylamine, then 6:3:1 ethyl acetate:hexane:triethylamine afforded (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate as an off-white solid (9.64 g, 85%). ¹H NMR (400 MHz, CDCl₃) δ 7.35-7.33 (m, 1H), 7.17-7.12 (m, 4H), 6.92-6.87 (m, 3H), 6.08 (s, 2H), 5.63 (d, 1H), 5.24-5.21 (m, 1H), 5.10 (s, 2H), 5.05-5.01 (m, 1H), 4.94-4.91 (m, 1H), 4.23-4.19 (m, 1H), 4.08 (s, 2H), 3.99-3.95 (m 1H), 3.83-3.62 (m, 3H), 3.51-3.45 (m, 1H), 3.22-3.16 (m, 1H), 3.05-2.88 (m, 3H), 2.83-2.78 (m, 1H), 2.74 (s, 3H), 2.69-2.63 (m, 1H), 1.89-1.85 (m, 1H), 1.59-1.54 (m, 1H), 0.84 (d, 6H). APCI-LCMS m/z 780 (M+H).

### Example 1, Step 2

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl 4-[(tert-butoxycarbonyl)amino]butanoate

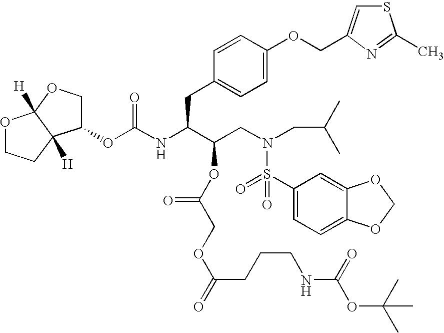

A solution of 4-(tert-butoxycarbonylamino)butyric acid (2.08 g, 10.3 mmol) in anhydrous tetrahydrofaran (60 mL) was treated with potassium t-butoxide (0.7 mL, 1.0 M in t-butanol). After stirring for 1.5 hours, the reaction was concentrated. Hexane was added and the mixture was concentrated again. To the resulting solid was added (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate (2.00 g, 2.56 mmol) and N,N-dimethylformamide (50 mL). The mixture was stirred at room temperature overnight, then diluted with water and extracted with dichloromethane. The organic layer was washed with water and saturated aqueous sodium chloride. After drying over magnesium sulfate, the solution was concentrated to a gold oil which solidified upon standing. Column chromatography on silica gel eluting with 97:3 chloroform:2M ammonia/methanol afforded a cream colored solid which contained a large quantity of N,N-dimethylformamide by ¹H NMR. This solid was dissolved in dichloromethane and washed with water (2×) and saturated aqueous sodium chloride, then dried over magnesium sulfate and concentrated to afford 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl 4-[(tert-butoxycarbonyl)amino]butanoate (2.17 g, 89%) as an off-white solid.

¹H NMR (300 MHz, DMSO-d₆) δ 7.38-7.34 (m, 1H), 7.19-7.14 (m, 4H), 6.94-6.88 (m, 3H), 6.10 (s, 2H), 5.65 (d, 1H), 5.31 (s, 1H), 5.28 (m, 1H), 5.12 (s, 2H), 5.07-5.00 (m, 2H), 4.73 (m, 1H), 4.62 (s, 2H), 4.20 (m, 1H), 4.00-3.95 (m, 1H), 3.85-3.65 (m, 3H), 3.49-3.43 (m, 1H), 3.22-3.17 (m, 3H), 3.07-2.78 (m, 4H), 2.76 (s, 3H), 2.68-2.60 (m, 1H), 2.50 (m, 2H), 1.92-1.83 (m, 3H), 1.45 (s, 9H), 0.86 (d, 6H). ES-LCMS m/z 947 (M+H).

### Example 1, Step 3

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl hydroxyacetate

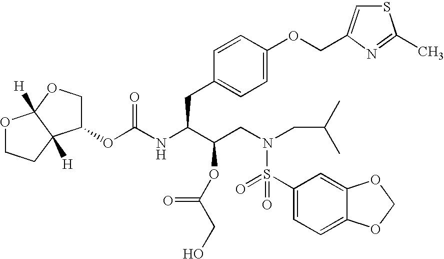

A solution of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl 4-[(tert-butoxycarbonyl)amino]butanoate (2.17 g, 22.9 mmol) in dichloromethane (25 mL) was treated with trifluoroacetic acid (10 mL) and stirred at room temperature for 1 hour. The reaction mixture was concentrated. Additional dichloromethane was added and concentrated (5×) to a gold oil. The oil was redissolved in dichloromethane and stirred with 10% aqueous potassium carbonate for 3 h, then dried over magnesium sulfate and concentrated. The crude product was purified by column chromatography on silica gel eluting with 97:3 chloroform:2M ammonia/methanol to afford (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl hydroxyacetate as an off-white foam (1.80 g, quantitative yield). ¹H NMR (300 MHz, DMSO-d₆) δ 7.51-7.45 (m, 2H), 7.36-7.29 (m, 2H), 7.15-7.09 (m, 3H), 6.91-6.88 (m, 2H), 6.18 (s, 2H), 5.50 (d, 1H), 5.34 (broad s, 1H), 5.13-5.10 (m, 1H), 5.02 (s, 2H), 4.89-4.83 (m, 1H), 4.06-3.82 (m, 3H), 3.71-3.38 (m, 5H), 3.19-3.07 (m, 1H), 2.94-2.67 (m, 5H), 2.64 (s, 3H), 1.85-1.79 (m, 1H), 1.35-1.28 (m, 1H), 1.13-1.07 (m, 1H), 0.80 (d, 6H). ES-LCMS m/z 762 (M+H).

### Example 1, Step 4

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl chloroacetate

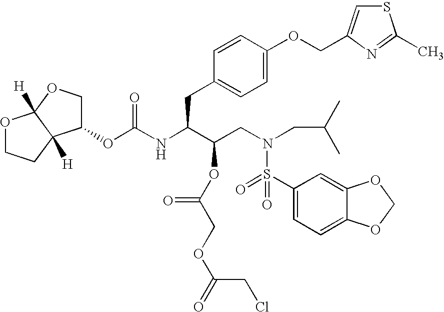

To a solution of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl hydroxyacetate (0.86 g, 1.1 mmol) and 4-(dimethylamino)pyridine (0.41 g, 3.4 mmol) in dichloromethane (17 mL) was added chloroacetyl chloride (0.25 mL, 3.1 mmol) dropwise over 1 minute. The reaction mixture was stirred for 0.5 hours, then diluted with water and 1N aqueous hydrochloric acid. The mixture was extracted with dichloromethane (2×). The organic layers were combined and washed with water and saturated aqueous sodium chloride. The solution was dried over magnesium sulfate, concentrated and triturated with diethyl ether to afford 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl chloroacetate as an off-white solid (0.80 g, 84%). ¹H NMR (400 MHz, CDCl₃) δ 7.34-7.31 (m, 1H), 7.16-7.12 (m, 4H), 6.91-6.86 (m, 3H), 6.07 (s, 2H), 5.62 (d, 1H), 5.27-5.25 (m, 1H), 5.10 (s, 2H), 5.01-4.96 (m, 2H), 4.72-4.67 (m, 2H), 4.20 (s, 2H), 4.18-4.16 (m, 1H), 3.98-3.94 (m, 1H), 3.82-3.65 (m, 3H), 3.44-3.39 (m, 1H), 3.24-3.18 (m, 1H), 3.03-2.98 (m, 1H), 2.93-2.77 (m, 3H), 2.73 (s, 3H), 2.66-2.60 (m, 1H), 1.87-1.83 (m, 1H), 1.59-1.53 (m, 1H), 1.41-1.35 (m, 1H), 0.85-0.82 (m, 6H). APCI-LCMS m/z 838 (M+H).

### Example 1, Step 5

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(2-methoxyethyl)-N-methylglycinate

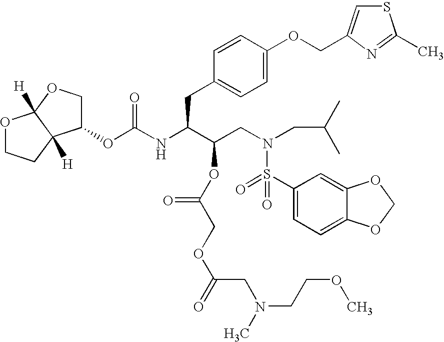

A solution of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl chloroacetate (62 mg, 0.074 mmol) in N,N-dimethylformamide (2 mL) was treated with N-(2-methoxyethyl)methylamine (22 μL, 0.22 mmol). The reaction mixture was stirred at room temperature for 3 hours, then diluted with water and extracted with ethyl acetate (2×). The organic layers were combined and washed with saturated aqueous sodium chloride, dried over magnesium sulfate and concentrated. The crude product was purified by column chromatography on silica gel eluting with 97:3 chloroform:2M ammonia/methanol to afford 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(2-methoxyethyl)-N-methylglycinate as a waxy solid. Lyophilization from acetonitrile and water produced a white solid (43 mg, 65%). ¹H NMR (400 MHz, DMSO-d₆) δ 7.50-7.47 (m, 2H), 7.34-7.27 (m, 2H), 7.13-7.07 (m, 3H), 6.89-6.87 (m, 2H), 6.16 (s, 2H), 5.48 (d, 1H), 5.11-5.09 (m, 1H), 5.01 (s, 2H), 4.87-4.82 (m, 1H), 4.70-4.58 (m, 2H), 3.91-3.81 (m, 2H), 3.68-3.49 (m, 4H), 3.40-3.37 (m, 4H), 3.19 (s, 3H), 3.15-3.09 (m, 1H), 2.91-2.65 (m, 7H), 2.62 (s, 3H), 2.31 (s, 3H), 1.83-1.76 (m, 1H), 1.33-1.27 (m, 1H), 1.10-1.05 (m, 1H), 0.83-0.78 (m, 6H). APCI-LCMS m/z 891 (M+H).

### EXAMPLE 2

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methyl-N-(2-pyridin-2-ylethyl)glycinate

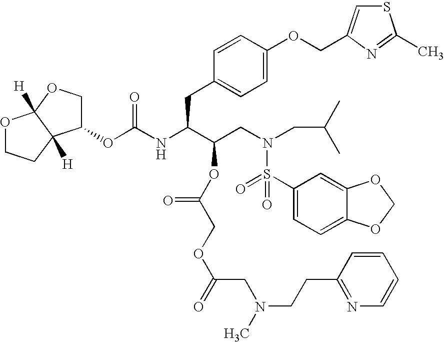

In a similar manner as described in example 1, step 5, from 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl chloroacetate (158 mg, 0.188 mmol) and 2-(2-methylaminoethyl)pyridine (78 μL, 0.56 mmol) was obtained 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methyl-N-(2-pyridin-2-ylethyl)glycinate (74 mg, 42%) as a white solid, except that a gradient from 5 to 40% isopropanol in chloroform was used for the chromatographic purification. ¹H NMR (400 MHz, DMSO-d₆) δ 8.49-8.47 (m, 1H), 7.72-7.67 (m, 1H), 7.54-7.52 (m, 2H), 7.38-7.30 (m, 3H), 7.23-7.11 (m, 4H), 6.94-6.91 (m, 2H), 6.20 (s, 2H), 5.53 (d, 1H), 5.15-5.13 (m, 1H), 5.05 (s, 2H), 4.93-4.86 (m, 1H), 4.77-4.63 (m, 2H), 3.92-3.85 (m, 2H), 3.74-3.42 (m, 6H), 3.21-3.13 (m, 1H), 2.96-2.70 (m, 8H), 2.67 (s, 3H), 2.45 (m, 1H), 2.40 (s, 3H), 1.86-1.82 (m, 1H), 1.39-1.09 (m, 2H), 0.85-0.81 (m, 6H). APCI-LCMS m/z 938 (M+H).

### EXAMPLE 3

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-diethylglycinate

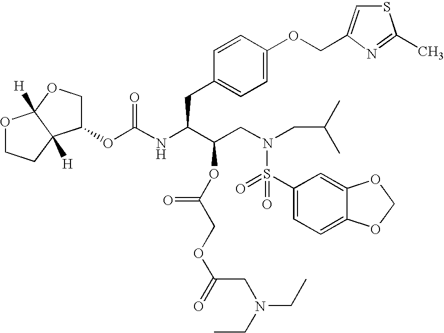

In a similar manner as described in example 1, step 5, from 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl chloroacetate (128 mg, 0.150 mmol) and diethylamine (47 μL, 0.46 mmol) was obtained 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-diethylglycinate (96 mg, 72%) as a white solid, except that a gradient from 5 to 10% isopropanol in chloroform was used for the chromatographic purification. ¹H NMR (400 MHz, DMSO-d₆) δ 7.49-7.43 (m, 2H), 7.33-7.30 (m, 1H), 7.26 (m, 1H), 7.11-7.06 (m, 3H), 6.88-6.86 (m, 2H), 6.15 (s, 2H), 5.47 (d, 1H), 5.10-5.09 (m, 1H), 5.00 (s, 2H), 4.86-4.81 (m, 1H), 4.63-4.61 (m, 1H), 3.90-3.80 (m, 3H), 3.68-3.64 (m, 1H), 3.58-3.48 (m, 3H), 3.38-3.20 (m, 4H, overlapping water signal), 3.15-3.08 (m, 1H), 2.90-2.64 (m, 5H), 2.62 (s, 3H), 2.59-2.51 (m, 4H), 2.42-2.41 (m, 1H), 1.79-1.77 (m, 1H), 1.31-1.26 (m, 1H), 1.09-1.04 (m, 1H), 0.96-0.92 (m, 3H), 0.79-0.77 (m, 6H). APCI-LCMS m/z 875 (M+H).

### EXAMPLE 4

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl morpholin-4-ylacetate

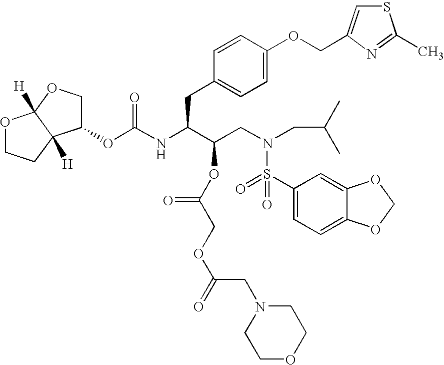

In a similar manner as described in example 1, step 5, from 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl chloroacetate (128 mg, 0.150 mmol) and morpholine (40 μL, 0.46 mmol) was obtained 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl morpholin-4-ylacetate (118 mg, 87%) as a white solid, except that 5% isopropanol in chloroform was used for the chromatographic purification. ¹H NMR (400 MHz, DMSO-d₆) δ 7.49-7.46 (m, 2H), 7.33-7.27 (m, 2H), 7.12-7.06 (m, 3H), 6.89-6.86 (m, 2H), 6.15 (s, 2H), 5.47 (d, 1H), 5.11-5.07 (m, 1H), 5.00 (s, 2H), 4.86-4.81 (m, 1H), 4.70-4.59 (m, 2H), 3.90-3.81 (m, 2H), 3.68-3.64 (m, 1H), 3.58-3.49 (m, 6H), 3.40-3.25 (m, 5H, overlapping water signal), 3.20-3.09 (m, 1H), 2.90-2.64 (m, 5H), 2.62 (s, 3H), 2.57-2.40 (m, 2H, overlapping dimethylsulfoxide signal), 1.82-1.75 (m, 1H), 1.32-1.21 (m, 1H), 1.09-1.05 (m, 1H), 0.79-0.77 (m, 6H), ES-LCMS m/z 889 (M+H).

### EXAMPLE 5

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-bis(2-methoxyethyl)glycinate

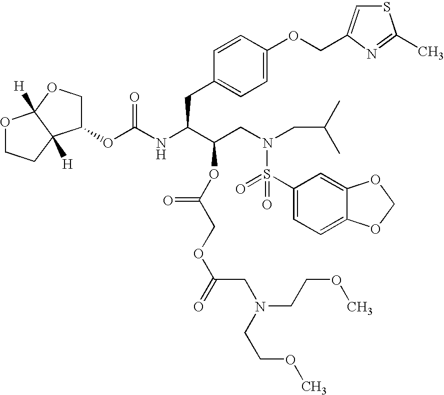

In a similar manner as described in example 1, step 5, from 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl chloroacetate (128 mg, 0.150 mmol) and bis(2-methoxyethyl)amine (68 μL, 0.46 mmol) was obtained 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-bis(2-methoxyethyl)glycinate (120 mg, 84%) as a white solid, except that 5% isopropanol in chloroform was used for the chromatographic purification. ¹H NMR (400 MHz, DMSO-d₆) δ 7.49-7.47 (m, 2H), 7.32-7.26 (m, 2H), 7.12-7.06 (m, 3H), 6.91-6.86 (m, 2H), 6.15 (s, 2H), 5.47 (d, 1H), 5.10-5.07 (m, 1H), 5.00 (s, 2H), 4.86-4.81 (m, 1H), 4.68-4.56 (m, 2H), 3.90-3.80 (m, 2H), 3.68-3.64 (m, 1H), 3.58-3.48 (m, 4H), 3.40-3.25 (m, 6H, overlapping water signal), 3.17 (m, 5H), 3.14-3.08 (m, 1H), 2.90-2.81 (m, 3H), 2.77 (m, 4H), 2.71-2.64 (m, 1H), 2.62 (s, 3H), 2.44-2.40 (m, 1H), 1.82-1.75 (m, 1H), 1.34-1.24 (m, 1H), 1.09-1.04 (m, 1H), 0.82-0.77 (m, 6H). ES-LCMS m/z 935 (M+H).

### EXAMPLE 6

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-lysinate tris-trifluoroacetic acid salt

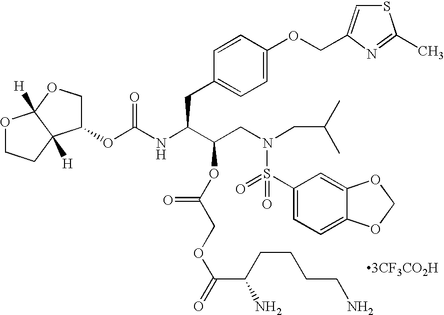

### Example 6, Step 1

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N²,N⁶-bis(tert-butoxycarbonyl)-L-lysinate

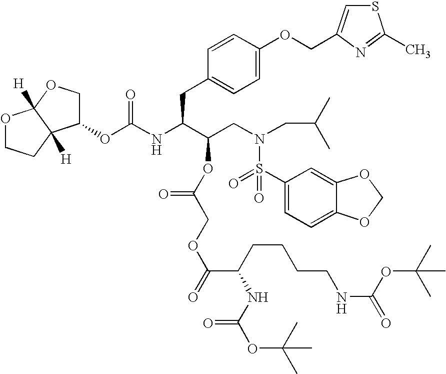

A solution of 0.100 g (0.128 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate (see example 1, step 1) and 0.202 g (0.384 mmol) of N²,N⁶-bis(tert-butoxycarbonyl)-L-lysine dicyclohexylamine salt in 4 mL of anhydrous N,N-dimethylformamide was heated to 100° C. with stirring. After 2 hours the solution was cooled to room temperature and diluted with ethyl acetate. The resulting solution was washed with saturated aqueous sodium chloride (4×), dried over magnesium sulfate, and concentrated to dryness at reduced pressure. The crude residue was purified by flash chromatography (silica gel, 95:5 dichloromethane/methanol) to afford 93 mg (66%) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N²,N⁶-bis(tert-butoxycarbonyl)-L-lysinate as a white foam. ¹H NMR (300 MHz, CDCl₃) δ 7.38 (dd, 1H), 7.27-7.11 (m, 4H), 7.02-6.83 (m, 3H), 6.12 (s, 2H), 5.68 (d, 1H), 5.53-4.92 (m, 7H), 4.83-4.52 (m, 3H), 4.41 (m, 1H), 4.20 (m, 1H), 4.00 (dd, 1H), 3.92-3.63 (m, 4H), 3.56-3.38 (m, 1H), 3.38-2.43 (m, 11H), 2.05-1.04 (m, 25H), 0.97-0.75 (m, 6H).

### Example 6, Step 2

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-lysinate tris-trifluoroacetic acid salt

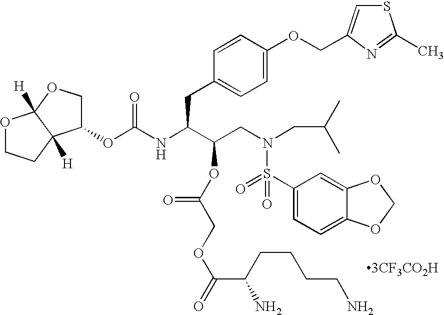

A solution of 93 mg (0.085 mmol) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N²,N⁶-bis(tert-butoxycarbonyl)-L-lysinate in 10 mL of 1:1 trifluoroacetic acid/dicloromethane was stirred at room temperature. After 1.5 hours analysis of the solution by LCMS indicated the reaction to be complete. The solution was concentrated to dryness at reduced pressure. The residue was dissolved in 6:4 water/acetonitrile, frozen, and lyophilized to afford 93 mg (89%) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-lysinate tris-trifluoroacetic acid salt as a light tan powder. ¹H NMR (300 MHz, DMSO-d₆) δ 8.60-8.41 (m, 3H), 7.74 (br s, 3H), 7.58-7.48 (m, 2H), 7.39-7.28 (m, 2H), 7.17-7.06 (m, 3H), 6.92 (d, 2H), 6.18 (s, 2H), 5.52 (d, 1H), 5.17 (m, 1H), 4.98-4.81 (m, 2H), 4.77 (d, 1H), 4.32-4.00 (m, 4H), 3.92-3.78 (m, 2H), 3.70 (t, 1H), 3.62-3.47 (m, 3H), 3.38 (d, 1H), 3.20(dd, 1H), 2.95-2.67 (m, 5H), 2.63 (s, 3H), 1.91-1.66 (m, 3H), 1.62-1.04 (m, 4H), 0.80 (d, 6H). ¹⁹F NMR (282 MHz, DMSO-d₆) δ −74.6 ppm. ES-LCMS m/z 890 (M+H).

### EXAMPLE 7

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-leucinate bis-trifluoroacetic acid salt

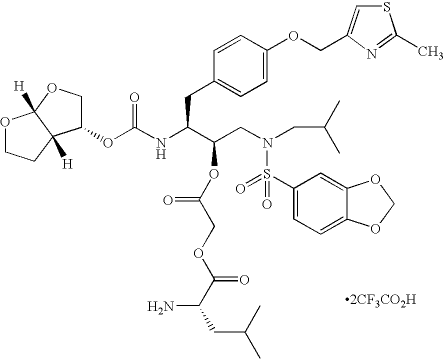

### Example 7, Step 1

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(tert-butoxycarbonyl)-L-leucinate

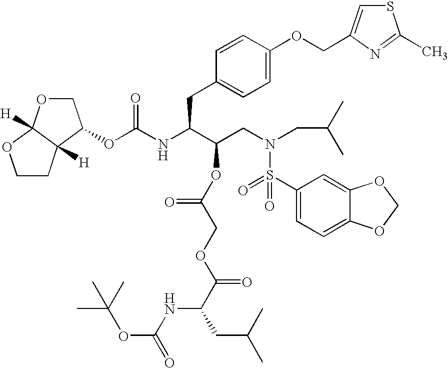

A solution of 0.128 g (0.512 mmol) of N-(tert-butoxycarbonyl)-L-leucine monohydrate (Bachem) in 10 mL of tetrahydrofuran was treated with 0.384 mL (0.384 mmol) of 1M potassium t-butoxide/t-butanol. A gelatinous precipitate resulted which was concentrated to dryness at reduced pressure. The residue was dissolved in 3 mL of anhydrous N,N-dimethylformamide and the solution added to 0.100 g (0.128 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate (see example 1, step 1). The resulting solution was heated to 70° C. with stirring. After 3 hours analysis by LCMS indicated the reaction to be complete. The mixture was cooled to room temperature and diluted with ethyl acetate. The resulting solution was washed with saturated aqueous sodium bicarbonate (2×), saturated aqueous sodium chloride (2×), dried over magnesium sulfate, and concentrated to dryness at reduced pressure. The crude material was subjected to flash chromatography (silica gel, 95:5 dichloromethane/methanol) to afford 97 mg (78%) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(tert-butoxycarbonyl)-L-leucinate as a white foam. ¹H NMR (300 MHz, CDCl₃) δ 7.33 (dd, 1H), 7.19-7.08 (m, 4H), 6.92-6.83 (m, 3H), 6.04 (s, 2H), 5.62 (d, 1H), 5.29 (m, 1H), 5.16-5.04 (m, 3H), 5.03-4.96 (m, 2H), 4.68 (d, 1H), 4.58 (d, 1H), 4.38 (m, 1H), 4.13 (m, 1H), 3.93 (dd, 1H), 3.80 (t, 1H), 3.76-3.65 (m, 2H), 3.43 (dd, 1H), 3.18 (dd, 1H), 3.04-2.72 (m, 4H), 2.72 (s, 3H), 2.62 (m, 1H), 1.92-1.66 (m, 3H), 1.62-1.48 (m, 2H), 1.45-1.34 (m, 10H), 0.93 (d, 6H), 0.83 (m, 6H).

### Example 7, Step 2

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-leucinate bis-trifluoroacetic acid salt

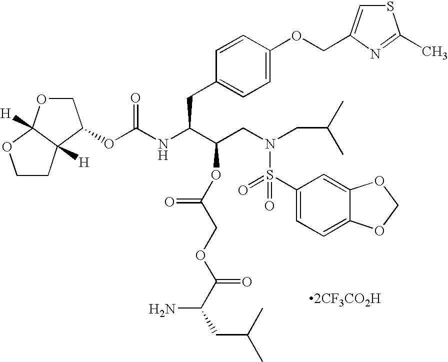

A solution of 97 mg (0.099 mmol) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(tert-butoxycarbonyl)-L-leucinate in 10 mL of 1:1 trifluoroacetic acid/dichloromethane was stirred at room temperature. After 2 hours analysis of the solution by LCMS indicated the reaction to be complete. The solution was concentrated to dryness at reduced pressure. The residue was dissolved in 6:4 water/acetonitrile, frozen, and lyophilized to afford 0.110 g (90%) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-leucinate bis-trifluoroacetic acid salt as a fluffy white solid. ¹H NMR (300 MHz, DMSO-d₆) δ 8.57-8.33 (m, 3H), 7.59-7.47 (m, 2H), 7.39-7.25 (m, 2H), 7.18-7.07 (m, 3H), 6.90 (d, 2H), 6.18 (s, 2H), 5.50 (d, 1H), 5.21-4.63 (m, 7H), 4.16 (br s, 1H), 3.96-3.80 (m, 2H), 3.73-3.48 (m, 3H), 3.39 (d, 1H), 3.19 (dd, 1H), 2.94-2.59 (m, 6H), 2.45 (m, 1H), 1.90-1.58 (m, 4H), 1.38-1.19 (m, 1H), 1.10 (dd, 1H), 0.91 (d, 6H), 0.80 (d, 6H).). ¹⁹F NMR (282 MHz, DMSO-d₆) δ −75.2 ppm. APCI-LCMS m/z 875 (M+H).

### EXAMPLE 8

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutylamino]methyl}-3-{4-[(2-methyl-1,3-thiazol-4-yl)methoxy]phenyl}propyl)oxy]-2-oxoethyl N-methylglycinate bis-trifluoroacetic acid salt

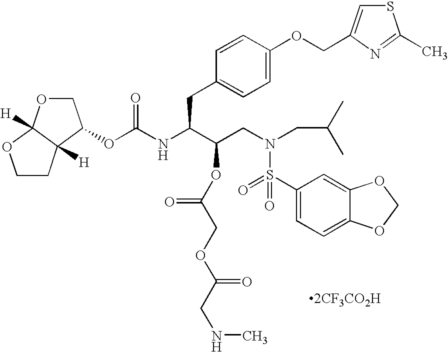

### Example 8, Step 1

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(tert-butoxycarbonyl)-N-methylglycinate

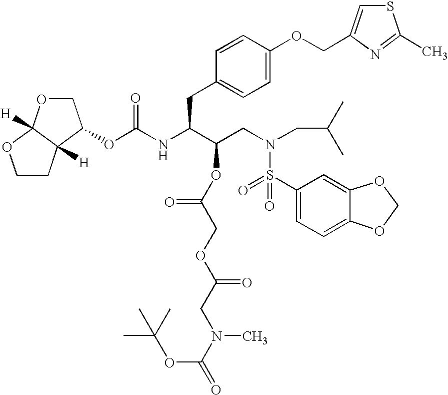

A solution of 97 mg (0.51 mmol) N-(tert-butoxycarbonyl)-N-methylglycine (Bachem) in 10 mL of tetrahydrofuran was treated with 0.384 mL (0.384 mmol) of 1M potassium t-butoxide/t-butanol. A gelatinous precipitate resulted which was concentrated to dryness at reduced pressure. The residue was dissolved in 3 mL of anhydrous N,N-dimethylformamide and the solution added to 0.100 g (0.128 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate (see example 1, step 1). The resulting solution was heated to 70° C. with stirring. After 3 hours analysis by LCMS indicated the reaction to be complete. The mixture was cooled to room temperature and diluted with ethyl acetate. The resulting solution was washed with saturated aqueous sodium bicarbonate (2×), saturated aqueous sodium chloride (2×), dried over magnesium sulfate, and concentrated to dryness at reduced pressure. The crude material was subjected to flash chromatography (silica gel, 95:5 dichloromethane/methanol) to afford 0.106 g (89%) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(tert-butoxycarbonyl)-N-methylglycinate as a white foam. ¹H NMR (300 MHz, CDCl₃) δ 7.33 (dd, 1H), 7.20-7.08 (m, 4H), 6.95-6.82 (m, 3H), 6.07 (s, 2H), 5.61 (d, 1H), 5.23 (br s, 1H), 5.17-4.94 (m, 3H), 4.72-4.60 (m, 2H), 4.18 (m, 1H), 4.08-3.90 (m, 2H), 3.85-3.60 (m, 3H), 3.47 (m, 1H), 3.18 (m, 1H), 3.07-2.56 (m, 13H), 1.95-1.20 (m, 12H), 0.90-0.79 (m, 6H). APCI-LCMS m/z 933 (M+H).

### Example 8, Step 2

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methylglycinate bis-trifluoroacetic acid salt

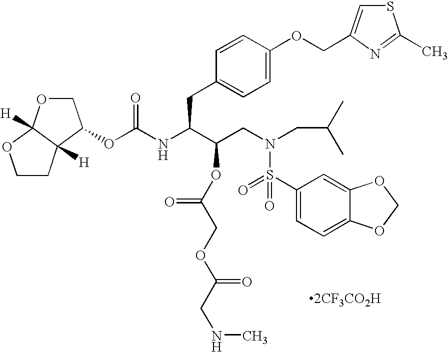

A solution of 0.106 g (0.114 mmol) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(tert-butoxycarbonyl)-N-methylglycinate in 10 mL of 7:3 trifluoroacetic acid/dichloromethane was stirred at room temperature. After 3 hours analysis of the solution by LCMS indicated the reaction to be complete. The solution was concentrated to dryness at reduced pressure. The residue was dissolved in 1:1 water/acetonitrile, frozen, and lyophilized to afford 0.119 g (98%) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methylglycinate bis-trifluoroacetic acid salt as a fluffy white solid. ¹H NMR (300 MHz, DMSO-d₆) δ 9.09 (br s, 2H), 7.59-7.48 (m, 2H), 7.40-7.28 (m, 2H), 7.18-7.07 (m, 3H), 6.91 (d, 2H), 6.19 (s, 2H), 5.52 (d, 1H), 5.36-4.65 (m, 6H), 4.17 (br s, 2H), 4.00-3.80 (m, 2H), 3.76-3.45 (m, 3H), 3.40 (d, 1H), 3.21 (dd, 1H), 2.96-2.39 (m, 11H), 1.80 (m, 1H), 1.31 (m, 1H), 1.11 (dd, 1H), 0.82 (d, 6H). ¹⁹F NMR (282 MHz, DMSO-d₆) δ −74.9 ppm. APCI-LCMS m/z 833 (M+H).

### EXAMPLE 9

### 2-[((1R,2S)-2-({[(3R,3aS,6aR)-hexahydrofuro[2,3-b]furan-3-yloxy ]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl[2-(2-ethoxyethoxy)ethoxy]acetate

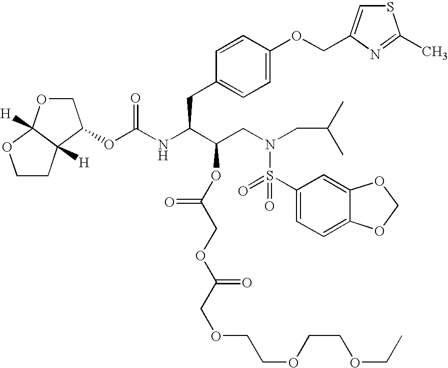

### Example 9, Step 1

### [2-(2-ethoxyethoxy)ethoxy]acetic acid

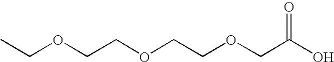

A mixture of tert-butyl chloroacetate (6.00 g, 40.0 mmol), 2-(ethoxyethoxy)ethanol (2.68 g, 20.0 mmol), 18-crown-6 (250 mg), and sodium hydroxide (12.0 g, 0.300 mol) in dioxane (60 mL) was stirred at room temperature for 24 hours. The dark mixture was diluted with water (250 mL) and twice extracted with ether (50 mL). The aqueous phase was acidified with 12N hydrochloric acid (35 mL) and extracted with dichloromethane (3×75 mL). The dichloromethane layers, after combining and concentrating under vacuum, were dried by dissolving in dichloromethane (50 ml) and concentrating under vacuum (3×) to afford [2-(2-ethoxyethoxy)ethoxy]acetic acid as a colorless oil (2.8 g, 73%). ¹H NMR (400 MHz, DMSO-d₆) δ 3.99 (s, 2H), 3.60-3.38 (m, 9H), 3.38 (q, 2H), 1.07 (t, 3H). ES-LCMS m/z 193 (M+H).

### Example 9, Step 2

### 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3thiazol-4-yl)methoxy]phenyl}propyl)oxy]-2-oxoethyl[2-(2-ethoxyethoxy)ethoxy]acetate

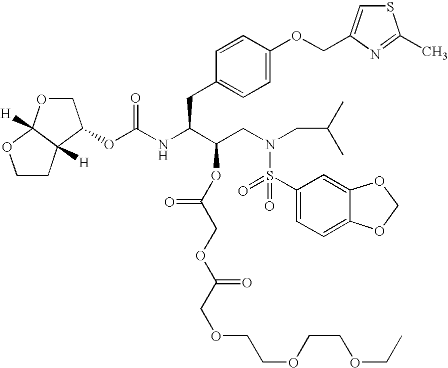

A solution of 1.18 g (6.16 mmol) of [2-(2-ethoxyethoxy)ethoxy]acetic acid in 20 mL of anhydrous tetrahydrofuran was treated with 4.62 mL (4.62 mmol) of 1M potassium t-butoxide/t-butanol. The resulting mixture was concentrated to dryness at reduced pressure. The residue was dissolved in 20 mL of anhydrous N,N-dimethylformamide and the solution added to 1.20 g (1.54 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate (see example 1, step 1). The resulting solution was heated to 70° C. with stirring. After 2 hours analysis by LCMS indicated the reaction to be complete. The mixture was cooled to room temperature and diluted with ethyl acetate. The resulting solution was washed with saturated aqueous sodium bicarbonate (3×), saturated aqueous sodium chloride (2×), dried over magnesium sulfate, and concentrated to dryness at reduced pressure. The crude material was subjected to flash chromatography (silica gel, 95:5 dichloromethane/methanol) to afford 0.91 g (63%) of 2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl[2-(2-ethoxyethoxy)ethoxy]acetate as a white foam. ¹H NMR (300 MHz, CDCl₃) δ 7.33 (d, 1H), 7.18-7.07 (m, 4H), 6.93-6.84 (m, 3H), 6.07 (s, 2H), 5.61 (d, 1H), 5.24 (m, 1H), 5.12-4.95 (m, 4H), 4.73-4.60 (m, 2H), 4.34-4.26 (m, 2H), 4.18 (m, 1H), 3.94 (m, 1H), 3.83-3.40 (m, 14H), 3.16 (dd, 1H), 3.04-2.82 (m, 3H), 2.81-2.68 (m, 4H), 2.60 (m, 1H), 1.85 (m, 1H), 1.54 (m, 1H), 1.37 (m, 1H), 1.18 (t, 3H), 0.90-0.77 (m, 6H). APCI-LCMS m/z 936 (M+H).

### EXAMPLE 10

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-(1H-imidazol-1-yl)ethoxy]acetate bis-trifluoroacetic acid salt

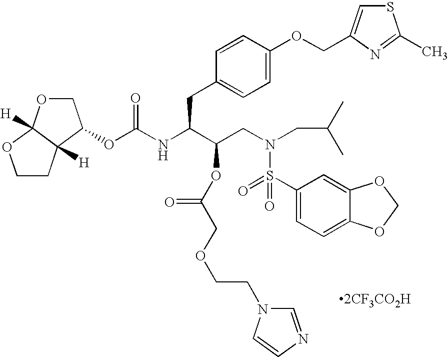

### Example 10, Step 1

### (2-chloroethoxy)acetic acid

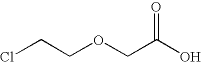

A solution of tert-butyl 2-(2-chloroethoxy)acetate (Hernandaz, Pedro E.; Fairfax, David E.; Michalson, Erik T. Process for preparing piperazine-substituted aliphatic carboxylates. PCT Int. Appl. (2001), WO 0129016 A1 20010426 CAN134:311229 AN 2001:300697) (1.00 g, 5.14 mmol) and concentrated hydrochloric acid (10 mL) was stirred for 2 hours. The reaction solution was diluted with water (20 mL) and extracted with ethyl ether (3×350 mL). The etheral layers were combined, washed with water (2×20 mL) and concentrated in a vacuum. The residue was dissolved in dichloromethane (60 mL) and concentrated in a vacuum (3×) to afford 2-(2-chloroethoxy)acetic acid as a clear oil (680 mg, 95%). ¹H NMR (400 MHz, DMSO-d₆) δ 8.4 (br s, 1H), 4.22 (s, 2H), 3.85 (t, 2H, J=5.9 Hz), 3.67 (t, 2H, J=5.9). ES-LCMS m/z 139 (M+H).

### Example 10, Step 2

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-chloroethoxy)acetate

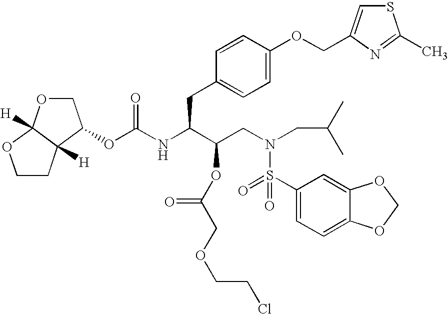

To solution of (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (2.13 mmol, 1.50 g), 2-(2-chloroethoxy)acetic acid (4.93 mmol, 0.680 g), and 4-(dimethylamino)pyridine (200 mg) in anhydrous N,N-dimethylformamide (15 mL) while stirring under a nitrogen atmosphere, was added N-[3-(dimethylamino)propyl]-N-ethylcarbodiimide hydrochloride (7.83 mmol, 1.50 g,) and stirring was continued for 24 hours. Chromatographic purification (silica gel, isopropanol/dichloromethane gradient, 0 to 15%) afforded (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl (2-chloroethoxy)acetate as a white foam (1.62 g, 92%). ¹H NMR (400 MHz, DMSO-d₆) δ 8.52-8.55 (m, 1H), 7.55-7.58 (m, 1H), 7.47-7.50 (m, 1H), 7.33-7.38 (m, 2H), 7.20-7.25 (m, 1H), 7.10-7.13 (m, 2H), 6.85-6.93 (m, 2H), 6.55-6.60 (m, 2H), 5.96 (s, 2H), 5.46-5.51 (m, 1H), 5.11 (s, 2H), 4.91-4.98 (m, 1H), 4.78-4.85 (m, 1H), 3.70-3.79 (m, 1H), 3.62-3.69 (m, 1H), 3.55-3.61 (m, 3H), 3.42-3.51 (m, 1H), 3.21-3.30 (m, 2H), 2.86-2.98 (m, 2H), 2.70-2.78 (m, 1H), 2.56-2.66 (m, 2H), 2.32-2.40 (m, 1H), 1.86-1.95 (m, 1H), 1.25-1.40 (m, 1H), 1.15-1.24 (m, 1H), 0.98-1.03 (m, 4H), 0.75-0.85 (m, 6H). ES-LCMS m/z 824 (M+H).

### Example 10, Step 3

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-iodoethoxy)acetate

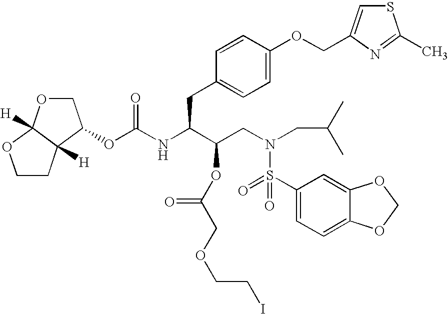

A mixture of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-chloroethoxy)acetate (7.52 mmol, 6.20 g) and sodium iodide (35 g) in acetone (100 mL) was refluxed with stirring under a nitrogen atmosphere for 2 days. The reaction mixture was concentrated in a vacuum and the residue was dissolved in water (100 mL) and extracted three times with ethyl acetate (75 mL). The organic layers were combined and extracted with water (3×20 mL). Removal of the volatiles by evaporation under vacuum afforded (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl (2-iodoethoxy)acetate as a white solid (5.36 g, 77%). ¹H NMR (400 MHz, DMSO-d₆) δ 7.74-7.53 (m, 2H), 7.29-7.37 (m, 2H), 7.08-7.17 (m, 3H), 6.88-6.94 (m, 2H), 6.19 (s, 2H), 5.49-5.52 (m, 1H), 5.10-5.16 (m, 1H), 5.04 (s, 2H), 4.84-4.92 (m, 1H), 4.02-4.23 (m, 2H), 3.90-3.98 (m, 1H), 3.83-3.88 (m, 1H), 3.65-3.80 (m, 3H), 3.52-3.63 (m, 2H), 3.33-3.45 (m, 4H), 3.15-3.25 (m, 1H), 2.86-2.94 (m, 1H), 2.76-2.84 (m, 2H), 2.66-2.75 (m, 1H), 2.65 (s, 3H), 1.77-1.86 (m, 1H), 1.28-1.42 (m, 1H), 1.10-1.20 (m, 1H), 0.76-0.82 (m, 6H). ES-LCMS m/z 915 (M+H).

### Example 10, Step 4

### (1R,2S)-2-({[(3R,3 aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-(1H-imidazol-1-yl)ethoxy]acetate bis-trifluroacetic acid salt

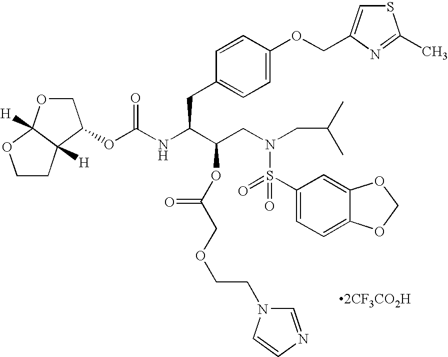

A solution of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-iodoethoxy)acetate (0.0109 mmol, 100 mg) and imidazole (2.23 mmol, 150 mg) in anhydrous dimethylsulfoxide (2 mL) was stirred for 24 hours. Chromatographic purification (Dynamax C₈ reverse phase chromatography column, 19 mm×300 mm, gradient from aqueous 1% trifluoroacetic acid to acetonitrile in 40 minutes, 20 mL/min) and lyophilization afforded (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-(1H-imidazol-1-yl)ethoxy]acetate bis-trifluoroacetic acid salt (85 mg, 62%) as a white powder. ¹H NMR (400 MHz, DMSO-d₆) δ 9.1 (bs, 1H), 7.66-7.80 (m, 2H), 7.44-7.55 (m, 2H), 7.26-7.38 (m, 2H), 7.06-7.18 (m, 3H), 6.84-6.96 (m, 2H), 6.18 (s, 2H), 5.48-5.54 (m, 1H), 5.06-5.18 (m, 1H), 5.10 (s, 2H), 4.82-4.92 (m, 1H), 4.36-4.44 (m, 2H), 4.00-4.24 (m, 1H), 3.82-3.94 (m, 3H), 3.10-3.75 (m, 6H), 2.66-2.90 (m, 4H), 2.62 (s, 3H), 2.25-2.40 (m, 2H), 1.16-1.19 (m, 2H), 1.23-1.43 (m, 1H), 1.03-1.20 (m, 1H), 0.63-0.90 (m, 6H). ES-LCMS m/z 856 (M+H).

### EXAMPLE 11

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{methyl[(6-methylpyridin-2-yl)methyl]amino}ethoxy)acetate tris-trifluoroacetic acid salt

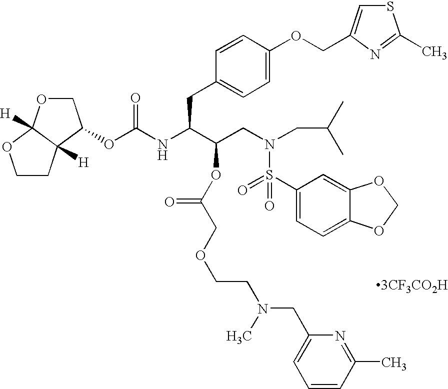

(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{methyl[(6-methylpyridin-2-yl)methyl]amino}ethoxy)acetate tris-trifluoroacetic acid salt was obtained as a lyophilized white solid (90 mg, 77% yield) from (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-iodoethoxy)acetate (100 mg, 0.109 mmol) N-methyl-1-(6-methylpyridin-2-yl)methanamine (300 mg, 2.20 mmol) following the procedure outlined in example 10 except that N-methyl-1-(6-methylpyridin-2-yl)methanamine was substituted for imidazole. ¹H NMR (400 MHz, DMSO-d₆) δ 7.77 (t, 1H, J=7.7 Hz), 7.45-7.50 (m, 2H), 7.2.5-7.31 (m, 4H), 7.06-7.12 (m, 3H), 6.86-6.90 (m, 2H), 6.15 (s, 2H), 5.47 (d, 1H, J=5.6 Hz), 5.10-5.17 (m, 1H), 5.0 (s, 2H), 4.79-4.83 (m, 1H), 4.47 (s, 2H), 4.02-4.24 (m, 2H), 3.79-3.98 (m, 5H), 3.60-3.65 (m 1H), 3.45-3.60 (m, 4H), 3.15-3.4 (m, 2H), 3.13 (s, 2H), 2.84 (s, 3H), 2.65-2.80 (m, 3H), 2.61 (s, 3H), 2.45 (s, 3H), 1.70-1.82 (m, 1H), 0.25-1.40 (m, 1H), 1.05-1.15 (m, 1H), 0.70-0.80 (m, 6H). ES-LCMS m/z 924 (M+H).

### EXAMPLE 12

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-oxo-2-(1H-pyrazol-5-ylamino)ethoxy]acetate

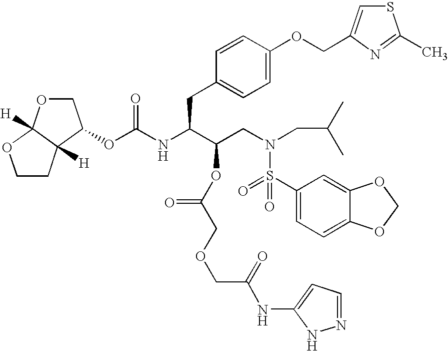

### Example 12, Step 1

### {2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethoxy}acetic acid

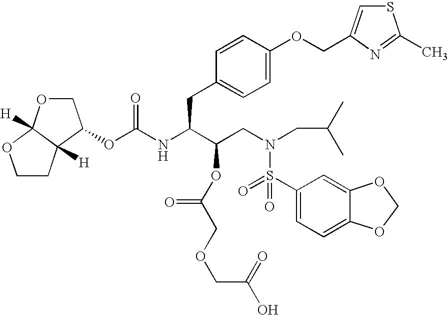

Diglycolic anhydride (2.44 g, 21.0 mmol) and N,N-diisopropylethylamine (3.66 mL, 21.0 mmol) were added to a solution of (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (4.92 g, 7.00 mmol) in dichloromethane (100 mL). The reaction mixture was stirred for 24 hours before the resulting brown solution was diluted with dichlormethane (250 mL). This solution was washed with saturated aqueous sodium hydrogen sulfate, and saturated aqueous sodium chloride. It was then dried over magnesium sulfate, and concentrated to a tan foam, which was largely redissolved in boiling ethyl acetate (800 mL). The hot cloudy suspension was then filtered through celite, and the filtrate was concentrated until an oil began to form. The mixture was then left to cool and stand overnight. The resulting solid was isolated by filtration, and dried under vacuum to afford {2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethoxy}acetic acid as a colorless solid (4.38 g, 76%). ¹H NMR (300 MHz, DMSO-d₆) δ 12.73 (br s, 1H), 7.54-7.51 (m, 2H), 7.38-7.32 (m, 2H), 7.18-7.11 (m, 3H), 6.94-6.91 (m, 2H), 6.21 (s, 2H), 5.53 (d, 1H), 5.18-5.10 (m, 1H), 5.05 (s, 2H), 4.91-4.85 (m, 1H), 4.26-4.04 (m, 4H), 4.01-3.93 (m, 1H), 3.91-3.85 (m, 1H), 3.75-3.68 (m, 1H), 3.65-3.53 (m, 2H), 3.50-3.42 (m, 1H), 3.28-3.13 (2H), 2.97-2.68 (m, 4H), 2.67 (s, 3H), 1.91-1.77 (m, 1H), 1.40-1.32 (m, 1H), 1.21-1.15 (m, 1H), 0.82 (d, 6H). ES-LCMS m/z 820 (M+H).

### Example 12, Step 2

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-oxo-2-(1H-pyrazol-5-ylamino)ethoxy]acetate

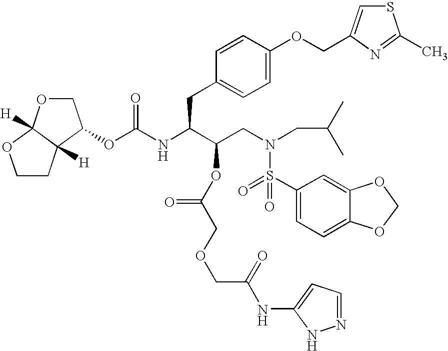

Polystyrene-supported carbodiimide (327 mg, 0.360 mmol) was added to 1.6 mL of a stock solution of {2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethoxy}acetic acid (295 mg, 0.360 mmol) and 1-hydroxybenzotriazole (54 mg, 0.40 mmol) in dichloromethane (3 mL) and N,N-dimethylformamide (1.8 mL). The resulting mixture was rocked at 800 Hz for 30 minutes, and then a solution of 3-aminopyrazole (15 mg, 0.18 mmol) in dichloromethane (1 mL) was added. The reaction mixture was then rocked at 800 Hz for 24 hours. Polystyrene-supported trisamine resin (107 mg, 0.260 mmol) and polystyrene-supported isocyanate resin (76 mg, 0.12 mmol) were then added, and the resulting mixture was rocked at 800 Hz for an additional 24 hours. The resins were removed by filtration, and washed with dichloromethane (30 mL). The washings and filtrate were combined and concentrated to afford a yellow oil, which was further purified by column chromatography on silica gel, eluting with a gradient from 2% to 4% methanol in dichloromethane to afford (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-oxo-2-(1H-pyrazol-5-ylamino)ethoxy]acetate as a colorless solid (45 mg, 42%). ¹H NMR (300 MHz, DMSO-d₆) δ 12.35 (br s, 1H), 10.02 (br s, 1H), 7.59 (br s, 1H), 7.60-7.48 (m, 2H), 7.37-7.27 (m, 2H), 7.14-7.08 (m, 3H), 6.90-6.87 (m, 2H), 6.48 (br s, 1H), 6.17 (s, 2H), 5.46 (d, 1H), 5.16-5.09 (m, 1H), 5.02 (s, 2H), 4.86-4.80 (m, 1H), 4.30-4.11 (m, 4H), 4.01-3.90 (m, 1H), 3.85-3.79 (m, 1H), 3.71-3.63 (m, 1H), 3.60-3.48 (m, 2H), 3.45-3.36 (m, 1H), 3.22-3.15 (m, 2H), 2.95-2.66 (m, 4H), 2.63 (s, 3H), 1.90-1.74 (m, 1H), 1.38-1.21 (m, 1H), 1.15-1.06 (m, 1H), 0.79 (d, 6H). ES-LCMS m/z 885 (M+H).

### EXAMPLE 13

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl{2-oxo-2-[(pyridin-2-ylmethyl)amino]ethoxy}acetate

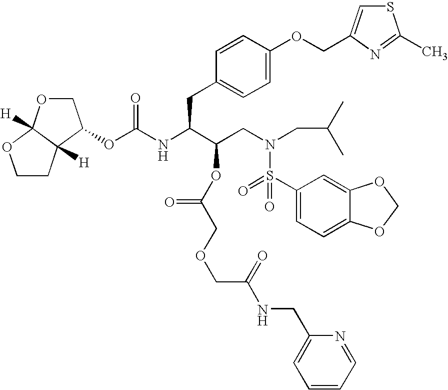

(1R,2S)-2-({[(3R,3aS,6aR)-Hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl {2-oxo-2-[(pyridin-2-ylmethyl)amino]ethoxy}acetate was obtained as a white solid (59 mg, 54%) from 2-aminomethylpyridine (19 μL, 0.18 mmol) and {2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethoxy}acetic acid (1.6 mL of a 75 mM solution in 5.4:1 dichloromethane:N,N-dimethylformamide), following the procedure outlined in example 12, step 2, except that a gradient from 3 to 4% methanol in dichloromethane was used for the chromatographic purification. ¹H NMR (300 MHz, DMSO-d₆) δ 8.50-8.47 (m, 1H), 8.39 (t, 1H), 7.76-7.70 (m, 3H), 7.51-7.48 (m, 2H), 7.35-7.23 (m, 4H), 7.14-7.08 (m, 3H), 6.91-6.88 (m, 2H), 6.17 (s, 2H), 5.49 (d, 1H), 5.19-5.10 (m, 1H), 5.02 (s, 2H), 4.88-4.62 (m, 1H), 4.41 (d, 2H), 4.28 (half of ABq, 1H), 4.14 (half of ABq, 1H), 4.03-3.90 (m, 1H), 3.88-3.81 (m, 1H), 3.72-3.64 (m, 1H), 3.60-3.49 (m, 2H), 3.43-3.34 (m, 1H), 3.26-3.16 (2H), 2.93-2.66 (m, 4H), 2.63 (s, 3H), 1.89-1.73 (m, 1H), 1.40-1.21 (m, 1H), 1.17-1.09 (m, 1H), 0.79 (d, 3H), 0.78 (d, 3H). ES-LCMS m/z 910 (M+H).

### EXAMPLE 14

### (1R,2S)-2-({[(3R,3aS,6aR)-Hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl {2-oxo-2-[(thien-2-ylmethyl)amino]ethoxy}acetate

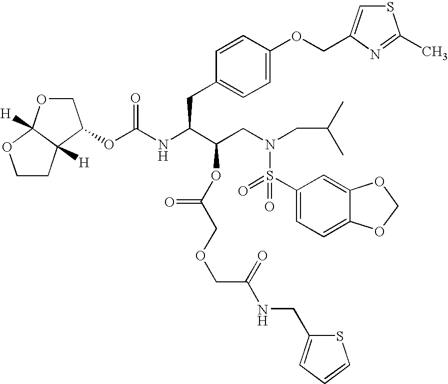

Polystyrene-supported carbodiimide (327 mg, 0.36 mmol) was added to a solution of {2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethoxy}acetic acid (98 mg, 0.12 mmol) and 1-hydroxybenzotriazole (21 mg, 0.16 mmol) in dichloromethane (2.0 mL) and N,N-dimethylformamide (0.6 mL). The resulting mixture was rocked at 800 Hz for 2 hours, and then 2-aminomethylthiophene (19 μL, 0.18 mmol) was added. The reaction mixture was rocked at 800 Hz for 24 hours, before the resin was removed via filtration and washed with dichloromethane (30 mL). The washings and filtrate were combined and concentrated to afford a yellow oil, which was further purified by column chromatography on silica gel, eluting with a gradient from 0% to 10% methanol in dichloromethane to afford (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl{2-oxo-2-[(thien-2-ylmethyl)amino]ethoxy}acetate

as a colorless foam (91 mg, 79%). ¹H NMR (300 MHz, DMSO-d₆) δ 8.43 (m, 1H), 7.51-7.47 (m, 2H), 7.38-7.27 (m, 3H), 7.15-7.07 (m, 3H), 6.97-6.86 (m, 4H), 6.17 (s, 2H), 5.45 (d, 1H), 5.15-5.08 (m, 1H), 5.02 (s, 2H), 4.88-4.82 (m, 1H), 4.46 (d, 2H), 4.24-4.00 (m, 5H), 3.99-3.89 (m, 1H), 3.87-3.82 (m, 1H), 3.72-3.65 (m, 1H), 3.61-3.50 (m, 2H), 3.41-3.35 (m, 1H), 3.24-3.19 (m, 1H), 2.94-2.64 (m, 4H), 2.64 (s, 3H), 1.90-1.72 (m, 1H), 1.40-1.22 (m, 1H), 1.19-1.07 (m, 1H), 0.79 (d, 3H), 0.78 (d, 3H). ES-LCMS m/z 915 (M+H).

### EXAMPLE 15

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{2-[methyl(2-pyridin-2-ylethyl)amino]-2-oxoethoxy}ethoxy)acetate

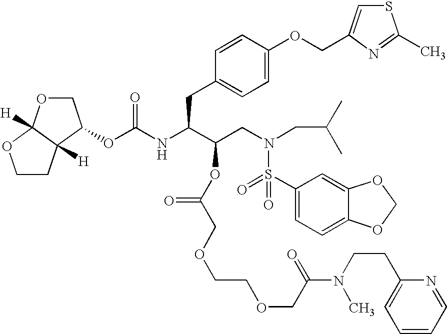

### Example 15, Step 1

### (10R)-10-((1S)-1-({[(3R,3aS,6aR)-hexahydrofuro[2,3-b]furan-3-yloxy]carbonyl}amino)-2-{4-[(2-methyl-1,3-thiazol-4-yl)methoxy]phenyl}ethyl)-12-(1,3-benzodioxol-5-ylsulfonyl)-14-methyl-8-oxo-3,6,9-trioxa-12-azapentadecan-1-oic acid

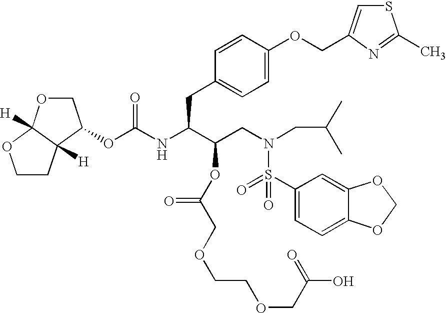

To a solution of 3,6-dioxaoctanedioic acid (0.531 g, 2.84 mmol) in 10 mL of anhydrous N,N-dimethylformamide was added 4-(dimethylamino)pyridine (0.092 g, 0.71 mmol) and 1-(3-dimethylaminopropyl)-3-ethylcarbodiimide hydrochloride (0.365 g, 1.78 mmol). The resulting solution was stirred for 30 minutes and then treated with (3R,3aS,6aR)-hexahydrofuro[2,3-b]furan-3-yl(1S,2R)-3-[(1,3-benzodioxol-5-ylsulfonyl)(isobutyl)amino]-2-hydroxy-1-{4-[(2-methyl-1,3-thiazol-4-yl)methoxy]benzyl}propylcarbamate (0.505 g, 0.711 mmol). After stirring for 24 hours the yellow solution was diluted with ethyl acetate (25 mL), washed with 5% aqueous sodium hydrogen sulfate, saturated aqueous sodium chloride, dried over magnesium sulfate and concentrated to afford a beige foam. The crude product was dissolved in acetonitrile (2 mL) and purified over two runs on C-8 reverse phase HPLC, eluting with a gradient from 20% to 80% acetonitrile in water (0.1% trifluoroacetic acid) over 40 minutes to afford (10R)-10-((1S)-1-({[(3R,3aS,6aR)-hexahydrofuro[2,3-b]furan-3-yloxy]carbonyl}amino)-2-{4-[(2-methyl-1,3-thiazol-4-yl)methoxy]phenyl}ethyl)-12-(1,3-benzodioxol-5-ylsulfonyl)-14-methyl-8-oxo-3,6,9-trioxa-12-azapentadecan-1-oic acid as a clear oil which was lyophilyzed to yield a white solid (0.322 g, 52%). ¹H NMR (400 MHz, DMSO-d₆) δ 7.50-7.44 (m, 2H), 7.33-7.26 (m, 2H), 7.13-7.06 (m, 2H), 6.90-6.86 (m, 2H), 6.15 (s, 2H), 5.49-5.46 (d, 1H), 5.12-5.06 (m, 1H), 5.02-4.99 (m, 2H), 4.87-4.81 (m, 1H), 4.16-4.07 (m, 2H), 3.96-3.80 (m, 3H), 3.69-3.48 (m, 9H), 3.40-3.33 (m, 1H), 3.20-3.11 (m, 1H), 2.90-2.64 (m, 5H), 2.49-2.45 (m, 4H), 1.84-1.75 (m, 1H), 1.36-1.25 (m, 1H), 1.13-1.06 (m, 1H), 0.80-0.74 (m, 6H). ES-LCMS m/z 864 (M+H).

### Example 15, Step 2

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{2-[methyl(2-pyridin-2-ylethyl)amino]-2-oxoethoxy}ethoxy)acetate

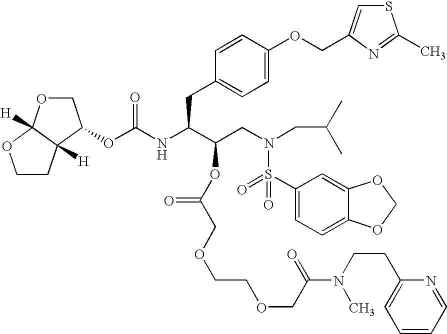

To a solution of (10R)-10-((1S)-1-({[(3R,3aS,6aR)-hexahydrofuro[2,3-b]furan-3-yloxy]carbonyl}amino)-2-{4-[(2-methyl-1,3-thiazol-4-yl)methoxy]phenyl}ethyl)-12-(1,3-benzodioxol-5-ylsulfonyl)-14-methyl-8-oxo-3,6,9-trioxa-12-azapentadecan-1-oic acid (1.50 g, 1.74 mmol) in 15 mL of anhydrous N,N-dimethylformamide was added 1.55 g (6.95 mmol) of carbonyl diimidazole. This was followed, after 2 hours, by addition of a second 1.53 g portion of carbonyl diimidazole. Following an additional 2 hour period the solution was treated with methyl[2-(2-pyridinyl)ethyl]amine (1.20 mL, 8.69 mmol). After stirring for 24 hours the reaction mixture was diluted with ethyl acetate (50 mL) and washed with saturated aqueous sodium chloride. It was then dried over magnesium sulfate and concentrated to a beige foam. The crude product was dissolved in acetonitrile (5 mL) and purified on C-8 reverse phase HPLC, eluting with a gradient from 30% to 100% acetonitrile in water (0.1% trifluoroacetic acid) over 40 minutes to afford (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{2-[methyl(2-pyridin-2-ylethyl)amino]-2-oxoethoxy}ethoxy)acetate as a clear oil which was lyophilyzed to yield a white solid (1.21 g, 68%). ¹H NMR (400 MHz, DMSO-d₆) δ 8.67-8.71 (m, 1H), 8.20-8.24 (m, 1H), 7.74-7.64 (m, 1H), 7.50-7.44 (m, 2H), 7.33-7.25 (m, 2H), 7.12-7.05 (m, 3H), 6.89-6.85 (m, 2H), 6.15 (s, 2H), 5.49-5.45 (d, 1H), 5.12-5.06 (m, 1H), 4.99 (s, 2H), 4.87-4.80 (m, 1H), 4.14-3.93 (m, 8H), 3.85-3.79 (m, 1H), 3.69-3.42 (m, 8H), 3.38-3.30 (m, 1H), 3.10-3.03 (m, 2H), 2.89-2.81 (m, 3H), 2.79-2.73 (m, 4H), 2.61 (s, 3H), 2.43-2.40 (m, 1H), 1.81-1.74 (m, 1H), 1.35-1.26 (m, 1H), 1.14-1.07 (m, 1H), 0.80-0.73 (m, 6H). ES-LCMS m/z 982 (M+H).

### EXAMPLE 16

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl N-[amino(imino)methyl]glycinate tris-trifluoroacetic acid salt

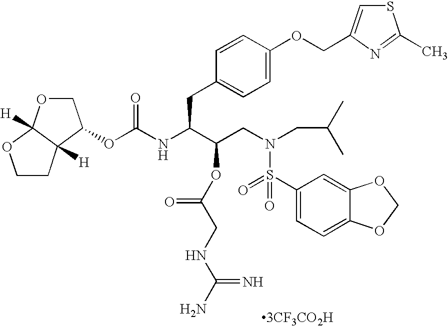

### Example 16, Step 1

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl azidoacetate

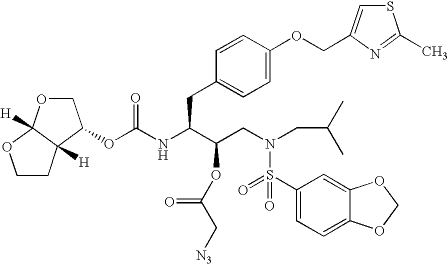

A solution of 2.00 g (2.56 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate (see example 1, step 1) and 0.330 g (5.12 mmol) of sodium azide in 15 mL of anhydrous N,N-dimethylformamide was stirred at room temperature for 3 hours. The resulting solution was diluted with ethyl acetate, washed with saturated aqueous sodium chloride (4×), dried over magnesium sulfate and concentrated at reduced pressure to give (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl azidoacetate as a light tan solid in quantitative yield. The intermediate was used without further purification. ¹H NMR (300 MHz, CDCl₃) δ 7.33 (dd, 1H), 7.18-7.08 (m, 4H), 6.95-6.84 (m, 3H), 6.08 (s, 2H), 5.63 (d, 1H), 5.28 (m, 1H), 5.09 (s, 2H), 5.06-4.92 (m, 2H), 4.22 (m, 1H), 4.00-3.68 (m, 5H), 3.63 (m, 1H), 3.48 (dd, 1H), 3.24 (dd, 1H), 3.07-2.77 (m, 4H), 2.75-2.61 (m, 4H), 1.87 (m, 1H), 1.58 (m, 1H), 1.34 (dd, 1H), 0.89-0.79 (m, 6H). APCI-LCMS m/z 787 (M+H).

### Example 16, Step 2

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl glycinate

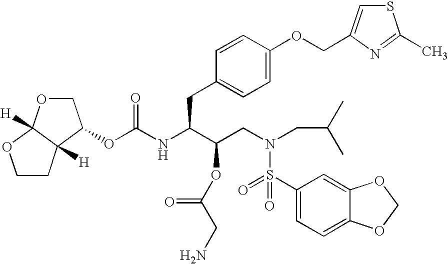

A solution of 2.06 g (2.61 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl azidoacetate in 60 mL of tetrahydrofuran was treated with 0.86 g (3.26 mmol) of triphenylphosphine followed by 5 mL of water. The resulting solution was stirred at room temperature for 18 hours and then concentrated to dryness at reduced pressure. The crude material was purified by flash chromatography (silica gel, 95:5 dichloromethane/2M ammonia in methanol) to afford 1.80 g (90%) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl glycinate as a white foam. ¹H NMR (300 MHz, DMSO-d₆) δ 7.58-7.41 (m, 2H), 7.40-7.22 (m, 2H), 7.20-7.04 (m, 3H), 6.91 (d, 2H), 6.18 (s, 2H), 5.50 (d, 1H), 5.18-4.95 (m, 3H), 4.84 (q, 1H), 3.98-3.80 (m, 2H), 3.77-3.06 (m, 9H), 3.02-2.40 (m, 6H), 1.97-1.53 (m, 3H), 1.43-1.06 (m, 2H), 0.90-0.77 (m, 6H). APCI-LCMS m/z 761 (M+H).

### Example 16, Step 3

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl N-{(Z)-[(tert-butoxycarbonyl)amino][(tert-butoxycarbonyl)imino]methyl}glycinate

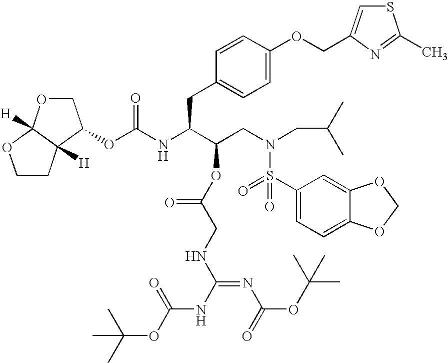

A solution of 0.200 g (0.263 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl glycinate, 0.113 g (0.289 mmol) of N,N′-bis(tert-butoxycarbonyl)-N″-triflylguanidine (Feichtinger, K., Sings, H. L., Baker, T. J., Matthews, K., Goodman, M. J. Org. Chem. 1998, 63, 8432) and 44 μL (0.32 mmol) of triethylamine in 4 mL of anhydrous dichloromethane was stirred at room temperature. After 18 hours the solution was diluted with ethyl acetate, washed with 10% aqueous sodium bisulfate (2×), water (2×), dried over magnesium sulfate and concentrated to dryness at reduced pressure. The crude product was subjected to flash chromatography (silica gel, 97:3 dichloromethane/methanol) to afford 0.158 g (60%) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl N-{(Z)-[(tert-butoxycarbonyl)amino][(tert-butoxycarbonyl)imino]methyl}glycinate as a white foam. ¹H NMR (300 MHz, DMSO-d₆) δ 11.87 (s, 1H), 8.68 (t, 1H), 7.55-7.43 (m, 2H), 7.39-7.29 (m, 2H), 7.19-7.05 (m, 3H), 6.90 (d, 2H), 6.18 (m, 2H), 5.51 (d, 1H), 5.13-4.98 (m, 3H), 4.87 (q, 1H), 4.18 (dd, 1H), 4.04-3.80 (m, 3H), 3.73-3.40 (m, 4H), 3.09 (dd, 1H), 3.01-2.82 (m, 2H), 2.80-2.61 (m, 5H), 2.46 (m, 1H, overlapping dimethylsulfoxide signal), 1.84 (m, 1H), 1.50 (s, 9H), 1.40-1.22 (m, 10H), 1.09 (m, 1H), 0.90-0.74 (m, 6H). ES-LCMS m/z 1003 (M+H).

### Example 16, Step 4

### (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-triazol-4-yl)methoxy]phenyl}propyl N-[amino(imino)methyl]glycinate tris-trifluoroacetic acid salt

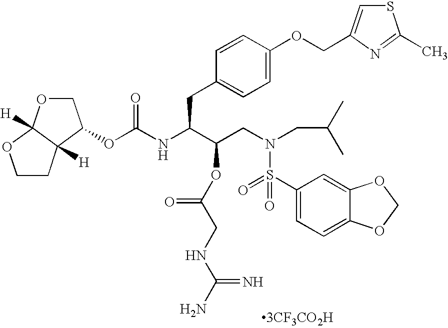

A solution of 40 mg (0.040 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl N-{(Z)-[(tert-butoxycarbonyl)amino][(tert-butoxycarbonyl)imino]methyl}glycinate in 10 mL of 8:2 trifluoroacetic acid/dichloromethane was stirred at room temperature for 2 hours. The solution was then evaporated to dryness at reduced pressure. The residue was dissolved 6:4 water/acetonitrile, frozen, and lyophilized to afford 42 mg (93%) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl N-[amino(imino)methyl]glycinate tris-trifluoroacetic acid salt as a white powder. ¹H NMR (300 MHz, DMSO-d₆) δ 7.70 (m, 1H), 7.58-7.04 (m, 10H), 6.90 (d, 2H), 6.19 (s, 2H), 5.51 (d, 1H), 5.14 (m, 1H), 5.03 (s, 2H), 4.88 (q, 1H), 4.19-3.92 (m, 4H), 3.87 (dd, 1H), 3.75-3.46 (m, 4H), 3.38 (m, 1H), 3.21 (dd, 1H), 2.95-2.68 (m, 4H), 2.66 (s, 3H), 2.45 (m, 1H, overlapping dimethylsulfoxide signal), 1.83 (m, 1H), 1.32 (m, 1H), 1.12 (m, 1H), 0.88-0.72 (m, 6H). ¹⁹F NMR (282 MHz, DMSO-d₆) δ −74.2 ppm. APCI-LCMS m/z 803 (M+H). HRMS m/z calcd. for C₃₆H₄₆N₆O₁₁S₂: 803.2744. Found: 803.2762.

### EXAMPLE 17

### 1-{2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl}pyridinium chloride

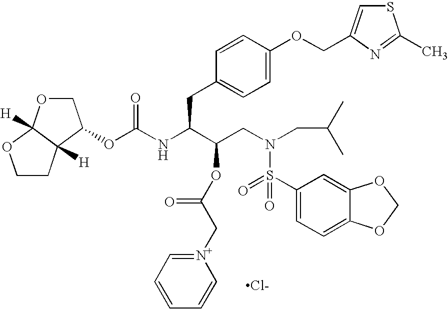

A solution of 0.250 g (0.320 mmol) of (1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl chloroacetate (see example 1, step 1) and 0.103 mL (1.28 mmol) of pyridine in 3 mL of dimethylsulfoxide was stirred at 80° C. in a sealed tube. After 18 hours the solution was cooled to room temperature, diluted with dichloromethane, washed with saturated aqueous sodium chloride (3×), dried over magnesium sulfate, and concentrated to dryness at reduced pressure to give a tan foam. This material was recrystallized twice from dichloromethane/ethyl acetate/hexane to afford 0.178 g (65%) of 1-{2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl}pyridinium chloride as an off-white powder. ¹H NMR (400 MHz, DMSO-d₆) δ 9.11-8.99 (m, 2H), 8.76 (t, 1H), 8.30 (t, 2H), 7.63 (d, 1H), 7.53 (s, 1H), 7.40-7.30 (m, 2H), 7.21-7.08 (m, 3H), 6.92 (d, 2H), 6.19 (s, 2H), 5.77 (ABq, 2H), 5.53 (d, 1H), 5.16 (m, 1H), 5.04 (s, 2H), 4.93 (q, 1H), 4.03 (m, 1H), 3.89 (dd, 1H), 3.72 (t, 1H), 3.62-3.51 (m, 2H), 3.29 (m, 2H, overlapping water signal), 2.93-2.72 (m, 4H), 2.68 (s, 3H), 2.48 (m, 1H, overlapping dimethylsulfoxide signal), 1.79 (m, 1H), 1.39 (m, 1H), 1.21 (m, 1H), 0.88-0.76 (m, 6H). ES-LCMS m/z 823 (M⁺). HRMS m/z calcd. for C₄₀H₄₇N₄O₁₁: 823.2683. Found: 823.2691. Elem. anal. calcd. for C₄₀H₄₇N₄O₁₁Cl.1.6H₂O: C, 54.09; H, 5.70; N, 6.31; Cl, 3.99. Found: C, 54.02; H, 5.68; N, 6.18; Cl, 3.84.

### EXAMPLE 18

### Pharmacokinetics in Sprague-Dawley Rats Following Single Oral Dose Employing Pre- and Co-Administration of Ritonavir

In order to assess the pharmacokinetic properties of the prodrugs of this invention, we administered single oral doses of a series of compounds of this invention, as well as the parent compound, GW0385, to male Sprague-Dawley rats. A dosing protocol using both pre- and co-administration of ritonavir was employed. The dose solutions of ritonavir were prepared by diluting the manufacturer's solution (80 mg/mL) 10-fold with a 50% aqueous solution of ethanol to a final concentration of 8 mg/mL. Each fasted animal received two doses of ritonavir by gavage (4 mg/kg/dose); the first approximately 30 minutes prior to dosing the test compound, and the second concurrently with the test compound. The prodrugs were administered at a dose of 3 mg/kg as solutions in 50% aqueous ethanol. Serial blood samples were collected through 8 hours post-dose and plasma concentrations of test compounds and the parent compound, GW0385, were determined by LC/MS/MS analysis. The estimated systemic exposures (area under the concentration-time curve extrapolated to infinity, AUC_{∞}) were determined by noncompartmental analysis of the plasma concentration-time curves. The Cₘₐₓ values (maximum observed concentration) were extracted from the individual concentration profiles. The key mean pharmacokinetic parameters, Cₘₐₓ and AUC_{∞} values, are given in table I.

TABLE I

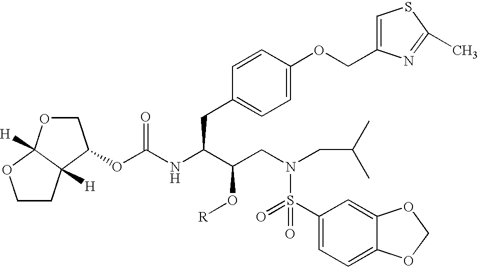

| Compound | | GW0385 Cₘₐₓ | GW0385 AUC | Prodrug Cₘₐₓ | Prodrug AUC |
|---|---|---|---|---|---|
| (Example) | R | (ng/mL) | (ng/mL · hr) | (ng/mL) | (ng/mL · hr) |
| GW0385 | H | 696 | 1960 | — | — |
| 1 | 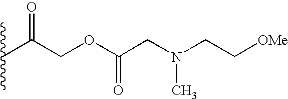 | 407 | 4713 | <1 | <1 |
| 2 | 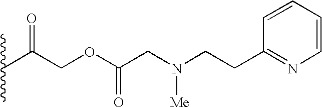 | 791 | 5353 | <1 | <1 |
| 3 | 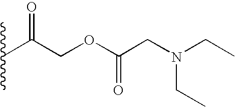 | 318 | 3693 | <1 | <1 |
| 4 | 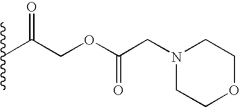 | 1279 | >9000 | <1 | <1 |
| 5 | 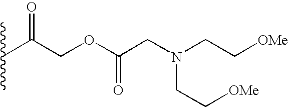 | 517 | >4000 | <1 | <1 |
| 6 | 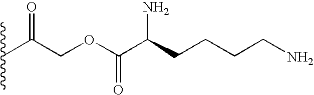 | 776 | 4220 | <1 | <1 |
| 7 | 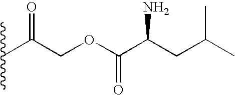 | 356 | 3957 | <1 | <1 |
| 8 | 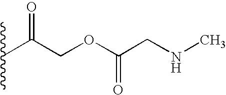 | 401 | 5601 | <1 | <1 |
| 9 | 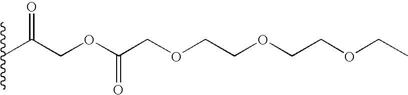 | 621 | >4000 | <1 | <1 |
| 10 | 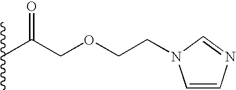 | 603 | >5000 | <1 | <1 |
| 11 | 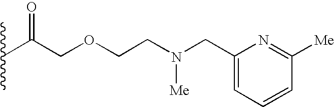 | 513 | 4017 | <1 | <1 |
| 12 | 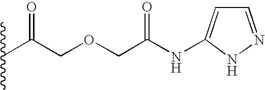 | 640 | 7035 | <1 | <1 |
| 13 | 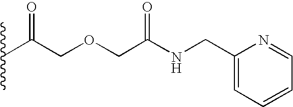 | 460 | 6807 | <1 | <1 |
| 14 | 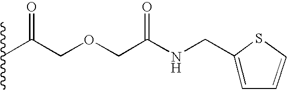 | 459 | 7286 | <1 | <1 |
| 15 | 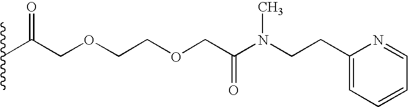 | 322 | 4215 | <1 | <1 |
| 16 | 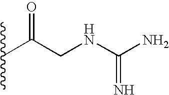 | 272 | >5000 | <1 | <1 |
| 17 | 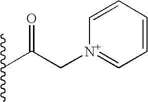 | 222 | >7700 | <1 | <1 |

The results demonstrate that oral administration of the prodrugs of this invention, along with pre- and co-dosing with ritonavir, resulted in significantly improved oral bioavailability in comparison to the parent compound GW0385. In addition, all prodrugs shown in table I underwent complete conversion to GW0385 as evidenced by the complete absence of detectable prodrug in the plasma.

What is claimed is:

1. A compounds of formula (I) 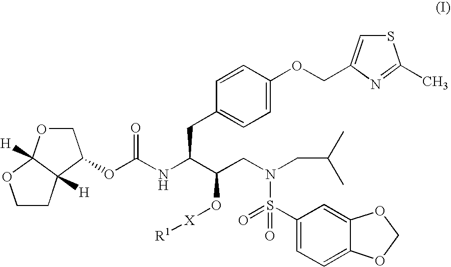
wherein:
X is a C₁₋₅ alkylene chain, wherein said C₁₋₅ alkylene chain is optionally substituted by one or more groups selected from ═O, ═N, —NH₂, and —C₁₋₈alkyl and wherein said C₁₋₅ alkylene chain optionally contains 1-4 heteroatoms selected from oxygen, sulfur and nitrogen wherein such heteroatom is optionally substituted with one or more groups selected from hydrogen and C₁₋₈alkyl;
R¹ is amino, C₁₋₈alkyl, C₁₋₈alkoxy, —NR², —N(R²)₂ or heterocycle optionally substituted with C₁₋₈alkyl;
R² is C₁₋₈alkyl or C₁₋₈alkoxy;
or a pharmaceutically acceptable salt thereof.

2. A compound of formula (I) according to claim 1 wherein —X—R¹ is selected from the group consisting of: 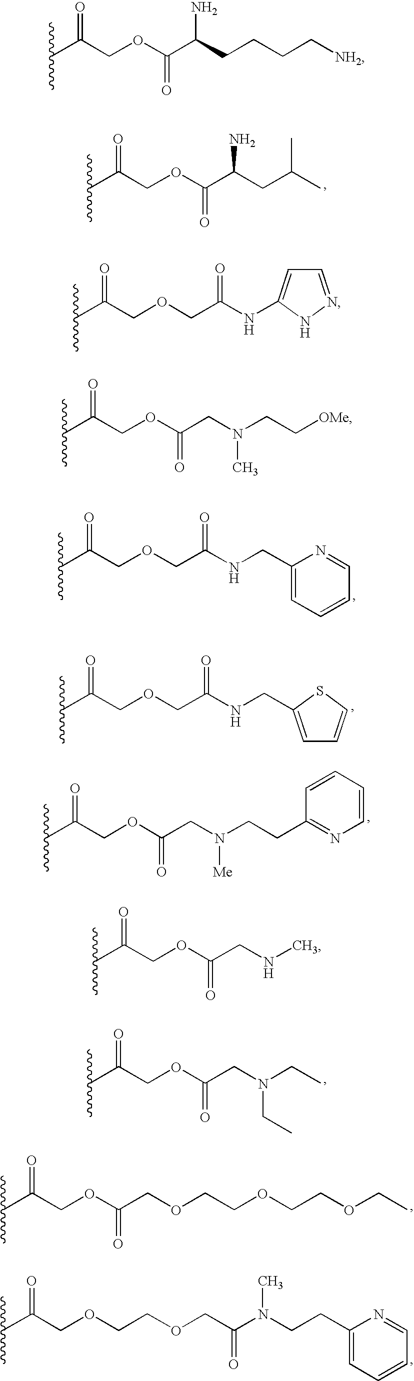 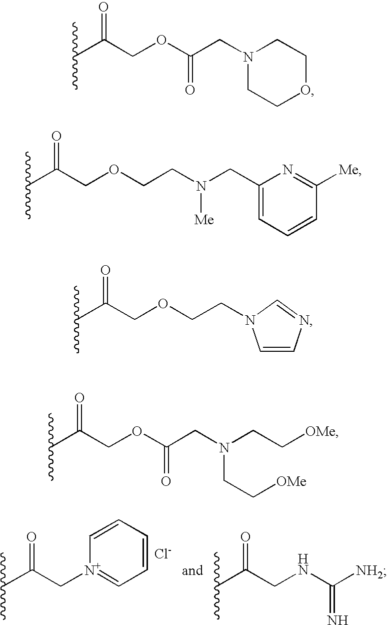
and pharmaceutically acceptable salts thereof.

3. A compound of formula (II) 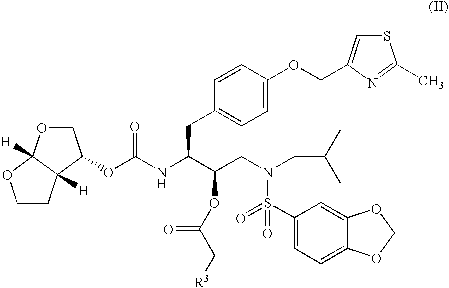
wherein:
R³ is hydroxy, halogen, aminoC₁₋₈alkyl, heterocycle, heterocycle C₁₋₈alkyl, N(R⁴)R⁵, NHR⁵, NHR⁵, OR⁵, OR⁶R⁷, OC(O)R⁴, OC(O)R⁵, OC(O)R⁶, OC(O)R⁴NR⁴, OC(O)R⁴NHR⁴, OC(O)NR⁴OR⁴, OC(O)R⁴N(R⁴)R⁵, OC(O)R⁴NHC(O)OR⁴, OC(O)R⁴N(R⁴)C(O)OR⁴, OC(O)R⁴N(R⁵)R⁴OR⁴, OR⁴C(O)OH, OR⁴C(O)NHR⁷, OR⁶C(O)N(R⁴)R⁷, OR⁶OC(O)OH;
R⁴ is C₁₋₈alkyl;
R⁵ is C₁₋₈alkyl, optionally substituted with one or more substituents selecting from the group consisting of amino, ═NH, N₃, halogen, oxo, C₁₋₈alkoxy, heterocycle, heterocyclealkyl, or NHC(O)R⁴; each of which may be optionally substituted with C₁₋₈alkyl;
R⁶ is C₁₋₅ alkylene chain, wherein said C₁₋₅ alkylene chain is optionally substituted by one or more groups selected from ═O, ═N, —NH₂, and —C₁₋₈alkyl and wherein said C₁₋₅ alkylene chain optionally contains 1-4 heteroatoms selected from oxygen, sulfur and nitrogen wherein such heteroatom is optionally substituted with one or more groups selected from hydrogen and C₁₋₈alkyl;
R⁷ is heterocycle optionally substituted with C₁₋₈alkyl or heterocycleC₁₋₈alkyl;
or a pharmaceutically acceptable salt thereof.

4. A compound selected from the group consisting of:
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-(2-methoxyethyl)-N-methylglycinate;
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methyl-N-(2-pyridin-2-ylethyl)glycinate;
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-diethylglycinate;
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl morpholin-4-ylacetate;
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N,N-bis(2-methoxyethyl)glycinate;
2-[((1R,2S)-2-({[(3R,3aS,6aR)-hexahydrofuro[2,3-b]furan-3-yloxy]carbonylamino}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-lysinate tris-trifluoroacetic acid salt;
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl L-leucinate bis-trifluoroacetic acid salt;
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl N-methylglycinate bis-trifluoroacetic acid salt;
(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-(1H-imidazol-1-yl)ethoxy]acetate bis-trifluoroacetic acid salt;
2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl[2-(2-ethoxyethoxy)ethoxy]acetate;
(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{methyl[(6-methylpyridin-2-yl)methyl]amino}ethoxy)acetate tris-trifluoroacetic acid salt;
(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl[2-oxo-2-(1H-pyrazol-5-ylamino)ethoxy]acetate;
(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl{2-oxo-2-[(pyridin-2-ylmethyl)amino]ethoxy}acetate;
(1R,2S)-2-({[(3R,3aS,6aR)-Hexahydrofuro[2,3-b]furan-3-yloxy]carbonyl}amino)-1-{[(1,3-benzodioxol-5-ylsulfonyl)(isobutyl)amino]methyl}-3-[4-[(2-methyl-1,3-thiazol-4-yl)methoxy]phenyl}propyl{2-oxo-2-[(thien-2-ylmethyl)amino]ethoxy)acetate;
(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl(2-{2-[methyl(2-pyridin-2-ylethyl)amino]-2-oxoethoxy}ethoxy)acetate;
(1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl N-[amino(imino)methyl]glycinate tris-trifluoroacetic acid salt; and
1-{2-[((1R,2S)-2-({[(3R,3aS,6aR)-hexahydrofuro[2,3-b]furan-3-yloxy]carbonyl}amino)-1-{[(1,3-benzodioxol-5-ylsulfonyl)(isobutyl)amino]methyl}-3-{4-[(2-methyl-1,3-thiazol-4-yl)methoxy]phenyl}propyl)oxy]-2-oxoethyl}pyridinium chloride;
and pharmaceutically acceptable salts thereof.

5. A method of treatment of a viral infection in a human comprising administering to said human an antiviral effective amount of a compound according to claim 1.

6. A method according to claim 5 wherein the viral infection is a HIV infection.

7. (canceled)

8. (canceled)

9. (canceled)

10. A pharmaceutical composition comprising an effective amount of a compound according to claim 1 together with a pharmaceutically acceptable carrier.

11. A pharmaceutical composition according to claim 10 in the form of a tablet or capsule.

12. A pharmaceutical composition according to claim 10 in the form of a liquid or suspension.

13. A method of treatment of a viral infection in a human comprising administering to said human a composition comprising a compound according to claim 1 and another therapeutic agent.

14. The method according to claim 13 wherein the viral infection is an HIV infection.

15. A composition according to claim 10, wherein said composition comprises at least one additional therapeutic agent selected from the group consisting of (1-alpha,2-beta,3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine (oxetanocin-G), TMC-114, BMS-232632, acyclic nucleosides [e.g. acyclovir, valaciclovir, famciclovir, ganciclovir, penciclovir), acyclic nucleoside phosphonates [e.g. (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid (tenofovir), (R)-[[2-(6-Amino-9H-purin-9-yl)-1-methylethoxy]methyl]phosphonic acid bis-(isopropoxycarbonyloxymethyl)ester (bis-POC-PMPA)], ribonucleotide reductase inhibitors (e.g. 2-acetylpyridine 5-[(2-chloroanilino)thiocarbonyl)thiocarbonohydrazone and hydroxyurea), nucleoside reverse transcriptase inhibitors (e.g., 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-Azido-2′,3′-dideoxythymidine-5′-H-phosphophonate (phosphonovir), 2′-deoxy-5-iodo-uridine (idoxuridine), as (−)-cis-1-(2-hydroxymethyl)-1,3-oxathiolane 5-yl)-cytosine (lamivudine), or cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin), protease inhibitors (e.g. indinavir, ritonavir, nelfinavir, amprenavir, saquinavir, (R)—N-tert-butyl-3-[(2S,3S)-2-hydroxy-3-N—[(R)-2-N-(isoquinolin-5-yloxyacetyl)amino-3-methylthiopropanoyl]amino-4-phenylbutanoyl]-5,5-dimethyl-1,3-thiazolidine-4-carboxamide (KNI-272), 4R-(4alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)-[N-(methoxycarbonyl)-I-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-Hydroxy-1(S)-indanyl)-2(R)-phenyl-methyl-4(S)-hydroxy-5-(1-(1-(4-benzo[b]furanylmethyl)-2(S)-N′-(tert-butylcarboxamido)piperazinyl)pentanamide (MK-944A), and GW 433908), interferons such as α-interferon, renal excretion inhibitors such as probenecid, nucleoside transport inhibitors such as dipyridamole; pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD₄ and genetically engineered derivatives thereof, non-nucleoside reverse transcriptase inhibitors (NNRTIs) for example, TMC-120, TMC-125, nevirapine (BI-RG-587), alpha-((2-acetyl-5-methylphenyl)amino)-2,6-dichloro-benzeneacetamide (loviride), 1-[3-(isopropylamino)-2-pyridyl]-4-[5-(methanesulfonamido)-1H-indol-2-ylcarbonyl]piperazine monomethanesulfonate (delavirdine), (10R,11S,12S)-12-Hydroxy-6,6,10,11-tetramethyl-4-propyl-11,12-dihydro-2H,6H,10H-benzo(1,2-b:3,4-b′:5,6-b″)tripyran-2-one ((+) calanolide A), (4S)-6-Chloro-4-[1E)-cyclopropylethenyl)-3,4-dihydro-4-(trifluoromethyl)-2(1H)-quinazolinone (DPC-083), 1-(ethoxymethyl)-5-(1-methylethyl)-6-(phenylmethyl)-2,4(1H,3H)-pyrimidinedione (MKC-442), 5-(3,5-dichlorophenyl)thio-4-isopropyl-1-(4-pyridyl)methyl-1H-imidazol-2-ylmethyl carbamate (capravirine), glycoprotein 120 antagonists [e.g. PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399)], cytokine antagonists [e.g. reticulose (Product-R), 1,1′-azobis-formamide (ADA), and 1,11-(1,4-phenylenebis(methylene))bis-1,4,8,11-tetraazacyclotetradecane octahydrochloride (AMD-3100)], and fusion inhibitors for example T-20 and T-124.

16. A method according to claim 13, wherein said therapeutic agent is selected from the group consisting of (1-alpha,2-beta,3-alpha)-9-[2,3-bis(hydroxymethyl)cyclobutyl]guanine [(−)BHCG, SQ-34514, lobucavir], 9-[(2R,3R,4S)-3,4-bis(hydroxymethyl)-2-oxetanosyl]adenine (oxetanocin-G), acyclic nucleosides [e.g. acyclovir, valaciclovir, famciclovir, ganciclovir, penciclovir), acyclic nucleoside phosphonates [e.g. (S)-1-(3-hydroxy-2-phosphonyl-methoxypropyl)cytosine (HPMPC), [[[2-(6-amino-9H-purin-9-yl)ethoxy]methyl]phosphinylidene]bis(oxymethylene)-2,2-dimethylpropanoic acid (bis-POM PMEA, adefovir dipivoxil), [[(1R)-2-(6-amino-9H-purin-9-yl)-1-methylethoxy]methyl]phosphonic acid (tenofovir), (R)-[[2-(6-Amino-9H-purin-9-yl)-1-methylethoxy]methyl]phosphonic acid bis-(isopropoxycarbonyloxymethyl)ester (bis-POC-PMPA)], ribonucleotide reductase inhibitors (e.g. 2-acetylpyridine 5-[(2-chloroanilino)thiocarbonyl)thiocarbonohydrazone and hydroxyurea), nucleoside reverse transcriptase inhibitors (e.g., 3′-azido-3′-deoxythymidine (AZT, zidovudine), 2′,3′-dideoxycytidine (ddC, zalcitabine), 2′,3′-dideoxyadenosine, 2′,3′-dideoxyinosine (ddI, didanosine), 2′,3′-didehydrothymidine (d4T, stavudine), (−)-beta-D-2,6-diaminopurine dioxolane (DAPD), 3′-Azido-2′,3′-dideoxythymidine-5′-H-phosphophonate (phosphonovir), 2′-deoxy-5-iodo-uridine (idoxuridine), as (−)-cis-1-(2-hydroxymethyl)-1,3-oxathiolane-5-yl)-cytosine (lamivudine), or cis-1-(2-(hydroxymethyl)-1,3-oxathiolan-5-yl)-5-fluorocytosine (FTC), 3′-deoxy-3′-fluorothymidine, 5-chloro-2′,3′-dideoxy-3′-fluorouridine, (−)-cis-4-[2-amino-6-(cyclopropylamino)-9H-purin-9-yl]-2-cyclopentene-1-methanol (abacavir), 9-[4-hydroxy-2-(hydroxymethyl)but-1-yl]-guanine (H2G), ABT-606 (2HM-H2G) and ribavirin), protease inhibitors (e.g. indinavir, ritonavir, nelfinavir, amprenavir, saquinavir, (R)—N-tert-butyl-3-[(2S,3S)-2-hydroxy-3-N—[(R)-2-N-(isoquinolin-5-yloxyacetyl)amino-3-methylthiopropanoyl]amino-4-phenylbutanoyl]-5,5-dimethyl-1,3-thiazolidine-4-carboxamide (KNI-272), 4R-(4alpha,5alpha,6beta)]-1,3-bis[(3-aminophenyl)methyl]hexahydro-5,6-dihydroxy-4,7-bis(phenylmethyl)-2H-1,3-diazepin-2-one dimethanesulfonate (mozenavir), 3-[1-[3-[2-(5-trifluoromethylpyridinyl)-sulfonylamino]phenyl]propyl]-4-hydroxy-6alpha-phenethyl-6beta-propyl-5,6-dihydro-2-pyranone (tipranavir), N′-[2(S)-Hydroxy-3(S)—[N-(methoxycarbonyl)-I-tert-leucylamino]-4-phenylbutyl-N^{alpha}-(methoxycarbonyl)-N′-[4-(2-pyridyl)benzyl]-L-tert-leucylhydrazide (BMS-232632), 3-(2(S)-Hydroxy-3(S)-(3-hydroxy-2-methylbenzamido)-4-phenylbutanoyl)-5,5-dimethyl-N-(2-methylbenzyl)thiazolidine-4(R)-carboxamide (AG-1776), N-(2(R)-Hydroxy-1(S)-indanyl)-2(R)-phenyl-methyl-4(S)-hydroxy-5-(1-(1-(4-benzo[b]furanylmethyl)-2(S)—N′-(tert-butylcarboxamido)piperazinyl)pentanamide (MK-944A), and GW 433908), interferons such as α-interferon, renal excretion inhibitors such as probenecid, nucleoside transport inhibitors such as dipyridamole, pentoxifylline, N-acetylcysteine (NAC), Procysteine, α-trichosanthin, phosphonoformic acid, as well as immunomodulators such as interleukin II or thymosin, granulocyte macrophage colony stimulating factors, erythropoetin, soluble CD₄ and genetically engineered derivatives thereof, non-nucleoside reverse transcriptase inhibitors (NNRTIs) [e.g. nevirapine (BI-RG-587), alpha-((2-acetyl-5-methylphenyl)amino)-2,6-dichloro-benzeneacetamide (loviride), 1-[3-(isopropylamino)-2-pyridyl]-4-[5-(methanesulfonamido)-1H-indol-2-ylcarbonyl]piperazine monomethanesulfonate (delavirdine), (10R,11S,12S)-12-Hydroxy-6,6,10,11-tetramethyl-4-propyl-11,12-dihydro-2H,6H,10H-benzo(1,2-b:3,4-b′:5,6-b″)tripyran-2-one ((+) calanolide A), (4S)-6-Chloro-4-[1E)-cyclopropylethenyl)-3,4-dihydro-4-(trifluoromethyl)-2(1H)-quinazolinone (DPC-083), 1-(ethoxymethyl)-5-(1-methylethyl)-6-(phenylmethyl)-2,4(1H,3H)-pyrimidinedione (MKC-442), 5-(3,5-dichlorophenyl)thio-4-isopropyl-1-(4-pyridyl)methyl-1H-imidazol-2-ylmethyl carbamate (capravirine)], glycoprotein 120 antagonists [e.g. PRO-2000, PRO-542 and 1,4-bis[3-[(2,4-dichlorophenyl)carbonylamino]-2-oxo-5,8-disodiumsulfanyl]naphthalyl-2,5-dimethoxyphenyl-1,4-dihydrazone (FP-21399)], cytokine antagonists [e.g. reticulose (Product-R), 1,1′-azobis-formamide (ADA), and 1,11-(1,4-phenylenebis(methylene))bis-1,4,8,11-tetraazacyclotetradecane octahydrochloride (AMD-3100)], and fusion inhibitors (e.g. T-20 and T-1249).

17. A method of treatment of a viral infection in a human comprising administering to said human an antiviral effective amount of a compound according to claim 3.

18. A method according to claim 17 wherein the viral infection is a HIV infection.

19. A pharmaceutical composition comprising an effective amount of a compound according to claim 2 together with a pharmaceutically acceptable carrier.
